(19) United States
(12) Patent Application Publication
(10) Pub. No.: US 2017/0239310 A1
(43) Pub. Date: Aug. 24, 2017

(54) Composition for Promoting Anti-Diabetic and Anti-Obesity Effects, Comprising Herbal Extract

(71) Applicant: DONGGUK UNIVERSITY GYEONGJU CAMPUS INDUSTRY- ACADEMY COOPERATION FOUNDATION, Gyeonsangbuk-do (KR)

(72) Inventors: Hojun Kim, Gyeonggi-do (KR); Young-Won Chin, Seoul (KR); Han Seok Choi, Seoul (KR)

(21) Appl. No.: 15/327,681

(22) PCT Filed: Jun. 29, 2015
(86) PCT No.: PCT/KR2015/006610

§ 371 Date: Jan. 20, 2017

(30) Foreign Application Priority Data
Jul. 21, 2014 (KR) 10-2014-0092194
Jul. 21, 2014 (KR) 10-2014-0092196
Jul. 21, 2014 (KR) 10-2014-0092197
Jun. 24, 2015 (KR) 10-2015-0089773
Jun. 24, 2015 (KR) 10-2015-0089774
Jun. 24, 2015 (KR) 10-2015-0089775

(51) Int. Cl. A61K 36/78; A61K 36/35; A61K 31/155; A61K 36/539

(52) U.S. Cl. CPC A61K 36/78; A61K 36/539; A61K 36/35; A61K 31/155

(57) ABSTRACT

The present invention relates to a composition for improving anti-diabetic and anti-obesity effects, including an extract extracted from any one selected from the group consisting of Lonicera japonica (Lonicerae Flos), Scutellaria baicalensis (Scutellariae Radix), and Houttuynia cordata (Houttuyniae Herba). According to the present invention, it was confirmed that co-administration of the extract of the present invention and metformin, a representative anti-diabetic drug, increases an anti-diabetic effect and reduces side effects caused by metformin, and at the same time, exhibits an anti-obesity effect by suppressing fat accumulation. Therefore, the composition of the present invention is expected to be effective in treatment of diabetes mellitus.

### TECHNICAL FIELD

The present invention relates to a composition for improving anti-diabetic and anti-obesity effects including the extract of a crude drug, and more particularly, to a composition including an extract of Lonicera japonica (Lonicerae Flos), Scutellaria baicalensis (Scutellariae Radix), or Houttuynia cordata (Houttuyniae Herba), which is capable of improving the therapeutic effects of metformin as an anti-diabetic drug on diabetes mellitus and simultaneously treating obesity.

### BACKGROUND ART

Diabetes mellitus is a disease characterized by high blood sugar, which is caused by absolute or relative insulin deficiency and insulin resistance in tissues, and metabolic disorders accompanying the same. Type 2 diabetes mellitus, which is increasing with the rise in obesity due to changes in dietary patterns and lifestyles as a result of development of human civilization, is attributed to insulin resistance considered as a major pathophysiological feature in type 2 diabetes mellitus, whereas type 1 diabetes mellitus results from absolute deficiency in insulin secretion. Along with genetic factors, insulin resistance is closely associated with dietary patterns responsible for reducing insulin sensitivity in peripheral tissues or a lifestyle including obesity, lack of exercise, stress, etc. Reduction of insulin sensitivity is highly correlated with obesity, which is supported by many studies demonstrating that insulin sensitivity is reduced when inflammatory responses occur in obese individuals.

Currently, as therapeutic agents for type 2 diabetes mellitus, there are sulfonylurea-class drugs responsible for increasing insulin secretion and antidiabetic drugs, such as pioglitazone and rosiglitazone, acting as peroxisome proliferator-activated receptor gamma (PPAR-γ) agonists responsible for improving insulin action. In addition, there are metformin class drugs responsible for reducing gluconeogenesis in the liver and acarbose-class drugs responsible for inhibiting digestion and absorption of carbohydrates, which prevents blood sugar from increasing after meals.

Among these drugs, metformin has the advantage of less side effects, such as hypoglycemia and weight gain, compared to other oral hypoglycemic agents, and thus is currently being used in primary pharmacotherapy for type 2 diabetic patients. At present, GLUCOPHAGE (a registered trademark of Bristol-Myers Squibb Company), which contains metformin hydrochloride as an active ingredient, is commercially available in tablet form. Each GLUCOPHAGE tablet contains 500, 850, or 1000 mg of metformin hydrochloride, and administration thereof is being implemented within the range not exceeding a maximum dose, i.e., 2,550 mg per day, considering the quantitative aspect of metformin related to drug efficacy and tolerance.

Although metformin, a major component of French lilac, has been used in Europe since 1957 and has been approved for use in America since 1994, the mechanism of action thereof has been revealed relatively recently. It has been reported, as a representative mechanism of action, that metformin inhibits gluconeogenesis in the liver and promotes fatty acid oxidation in the muscles and liver by inducing activation of AMP-activated protein kinase (AMPK), which is involved in regulation of cellular energy metabolism. Recent studies have shown that the action of metformin lowering blood sugar level is attributed to activation of LKB1, an upstream AMPK kinase (i.e., a kinase responsible for phosphorylating AMPK) and LKB1-mediated phosphorylation of TORC2, a transcriptional co-activator, is responsible for the inhibitory effect of metformin on gluconeogenesis.

However, it has been reported that 20 to 30% of patients taking metformin suffer side effects, such as loss of appetite, abdominal distension, nausea, and diarrhea. In addition, it has been reported that metformin rarely causes lactic acidosis, and thus attention should be paid when metformin is used for type 2 diabetic patients with renal disease, liver disease, hypoxia, severe infections, alcoholism, and the like. These side effects can be partially resolved by reducing minimum and/or sustained dosages, by reducing the number of doses, or by administering in combination with other drugs.

Accordingly, increasing the therapeutic effects of metformin on diabetes mellitus and decreasing the side effects of the same by combined or mixed use of metformin and other drugs have become a major research project, and thus related studies have been performed (e.g., Korea Patent No. 10-2011-0123908), but there is much to be studied.

### DISCLOSURE

### Technical Problem

Therefore, the present invention has been made to resolve the above problems. The present inventors have identified that combined use of metformin, an anti-diabetic drug, and the extract of Lonicera japonica (Lonicerae Flos), Scutellaria baicalensis (Scutellariae Radix) or Houttuynia cordata (Houttuyniae Herba) increases an anti-diabetic effect, decreases side effects and exhibits an inhibitory effect on fat accumulation, thereby completing the present invention.

Thus, it is an objective of the present invention to provide a pharmaceutical composition for improving an anti-diabetic effect, which is used in combination with metformin, an anti-diabetic drug, and includes an extract extracted from any one selected from the group consisting of Lonicera japonica (Lonicerae Flos), Scutellaria baicalensis (Scutellariae Radix), and Houttuynia cordata (Houttuyniae Herba).

However, the technical problems that are intended to be achieved in the present invention are not restricted to the above described problems, and other problems, which are not mentioned herein, could be clearly understood by those of ordinary skill in the art from details described below.

### Technical Solution

To achieve the objective of the present invention as described above, the present invention provides a pharmaceutical composition for improving an anti-diabetic effect, which is used in combination with metformin, an anti-diabetic drug, and includes an extract extracted from any one selected from the group consisting of Lonicera japonica (Lonicerae Flos), Scutellaria baicalensis (Scutellariae Radix), and Houttuynia cordata (Houttuyniae Herba).

According to one embodiment of the present invention, the pharmaceutical composition may be administrated simultaneously with or separately from metformin, the anti-diabetic drug, or the pharmaceutical composition and metformin may be administrated sequentially.

According to another embodiment of the present invention, the pharmaceutical composition may suppress differentiation of fat cells.

According to still another embodiment of the present invention, the extract may be extracted using one or more solvents selected from the group consisting of water, alcohols having 1 to 4 carbons, and a combination thereof.

According to yet another embodiment of the present invention, the pharmaceutical composition may increase expression levels of one or more selected from the group consisting of phosphorylated AMP-activated protein kinase (p-AMPK) and genes encoding sirtuin 1 (SirT1), AMP-activated protein kinase-alpha (AMPK-α), peroxisome proliferator-activated receptor-alpha (PPAR-α), and peroxisome proliferator-activated receptor-gamma (PPAR-γ), respectively.

According to yet another embodiment of the present invention, the pharmaceutical composition may decrease expression levels of one or more selected from the group consisting of genes encoding X-box binding protein 1 (XBP-1), tumor necrosis factor-alpha (TNF-α), and interleukin-6 (IL-6), respectively.

In addition, the present invention provides a method of improving an anti-diabetic effect and/or treating diabetes mellitus, the method including a step of administering an extract extracted from any one selected from the group consisting of Lonicera japonica (Lonicerae Flos), Scutellaria baicalensis (Scutellariae Radix), and Houttuynia cordata (Houttuyniae Herba) to individuals.

In addition, the present invention provides use of an extract extracted from any one selected from the group consisting of Lonicera japonica (Lonicerae Flos), Scutellaria baicalensis (Scutellariae Radix), and Houttuynia cordata (Houttuyniae Herba) to treat diabetes mellitus.

### Advantageous Effects

The composition according to the present invention includes an extract, as an active ingredient, extracted from any one selected from the group consisting of Lonicera japonica (Lonicerae Flos), Scutellaria baicalensis (Scutellariae Radix), and Houttuynia cordata (Houttuyniae Herba). It was confirmed that combined use of the extract and metformin, an anti-diabetic drug, improves therapeutic effects on diabetes mellitus and prediabetes and reduces side effects. Thus, it is expected that the extract can be usefully used as a pharmaceutical composition for improving a therapeutic effect on diabetes mellitus. In addition, it was confirmed that the extract exhibits an inhibitory effect on fat accumulation along with the therapeutic effect on diabetes mellitus. Therefore, it is expected that the extract can prevent or treat obesity along with treating diabetes.

### BRIEF DESCRIPTION OF DRAWINGS

FIG. 1 is a result showing the viability of 3T3-L1 cells dependent upon administration of each Lonicera japonica extract (GEH: water extract, GEH30: 30% ethanol extract, and GEH100: 100% ethanol extract).

FIG. 2 is a result showing the viability of 3T3-L1 cells dependent upon co-administration of a Lonicera japonica extract (GEH: water extract, GEH30: 30% ethanol extract, or GEH100: 100% ethanol extract) and metformin.

FIG. 3 is a result showing the viability of 3T3-L1 cells dependent upon administration of various concentrations (20, 50, 100, 200 μg/ml) of Lonicera japonica extracts.

FIG. 4 is a result showing changes in intracellular reactive oxygen species (ROS) activity in HepG2 cells dependent upon co-administration of a Lonicera japonica extract (GEH: water extract, GEH30: 30% ethanol extract, or GEH100: 100% ethanol extract) and metformin.

FIG. 5 is a result showing the inhibitory effects of administration of Lonicera japonica extracts (GEH: water extract, GEH30: 30% ethanol extract, GEH100: 100% ethanol extract) on nitrogen monoxide generation in RAW 264.7 cells.

FIG. 6 is a result showing the inhibitory effect of co-administration of a Lonicera japonica extract (GEH: water extract, GEH30: 30% ethanol extract or GEH100: 100% ethanol extract) and metformin on nitrogen monoxide generation in RAW 264.7 cells.

FIG. 7 is a result showing the suppressive effect of co-administration of a Lonicera japonica extract (GEH: water extract, GEH30: 30% ethanol extract, or GEH100: 100% ethanol extract) and metformin on fat cell differentiation in 3T3-L1 cells.

FIG. 8 is a result showing an increased glucose uptake capacity of undifferentiated L6 rat myoblast cells by co-administration of a Lonicera japonica 100% ethanol extract (GEH) and metformin.

FIG. 9 is a result showing the effect of co-administration of a Lonicera japonica extract (GEH: water extract, GEH30: 30% ethanol extract, or GEH100: 100% ethanol extract) and metformin on the insulin secretion capacity of RIN-m5F insulinoma cells.

FIG. 10 is a result showing whether insulin resistance is improved by co-administration of a Lonicera japonica extract and metformin (GEH+Met1) in undifferentiated L6 rat myoblast cells.

FIG. 11 is a result showing changes in the protein expression level of dipeptidyl peptidase-4 (DPP-4) by co-administration of a Lonicera japonica 100% ethanol extract (GEH100) and metformin in 3T3-L1 cells.

FIG. 12 is a result showing changes in the protein expression level of PPAR-γ by co-administration of a Lonicera japonica 100% ethanol extract (GEH100) and metformin in 3T3-L1 cells.

FIG. 13 is a result showing changes in the protein expression level of PPAR-γ by co-administration of each of the Lonicera japonica extracts at various concentrations (50, 100, 200 μg/ml) and metformin in 3T3-L1 cells.

FIG. 14 is a result showing changes in the protein expression levels of SirT1 and p-AMPK by administration of metformin (M), Lonicera japonica 30% ethanol extract (GEH) or a combination thereof (M+GEH) in RAW 264.7 cells.

FIG. 15 is a result showing changes in the gene expression level of AMPK-α by administration of metformin (M), the combination of a Lonicera japonica 30% ethanol extract and metformin (M+GEH) or the combination of a Lonicera japonica water extract and metformin (M+GEHW) in RAW 264.7 cells.

FIG. 16 is a result showing changes in the gene expression level of PPAR-α by administration of metformin (M), the combination of a Lonicera japonica 30% ethanol extract and metformin (M+GEH) or the combination of a Lonicera japonica water extract and metformin (M+GEHW) in RAW 264.7 cells.

FIG. 17 is a result showing changes in the gene expression level of PPAR-γ by administration of metformin (M), the combination of a Lonicera japonica 30% ethanol extract and metformin (M+GEH) or the combination of a Lonicera japonica water extract and metformin (M+GEHW) in RAW 264.7 cells.

FIG. 18 is a result showing changes in the gene expression level of XBP-1 by administration of metformin (M), the combination of a Lonicera japonica 30% ethanol extract and metformin (M+GEH) or the combination of a Lonicera japonica water extract and metformin (M+GEHW) in RAW 264.7 cells.

FIG. 19 is a result showing changes in the gene expression level of TNF-α by administration of metformin (M), the combination of a Lonicera japonica 30% ethanol extract and metformin (M+GEH) or the combination of a Lonicera japonica water extract and metformin (M+GEHW) in RAW 264.7 cells.

FIG. 20 is a result showing changes in the gene expression level of IL-6 by administration of metformin (M), the combination of a Lonicera japonica 30% ethanol extract and metformin (M+GEH) or the combination of a Lonicera japonica water extract and metformin (M+GEHW) in RAW 264.7 cells.

FIG. 21 indicates results showing (a) changes of insulin resistance and (b) changes in blood sugar level over time by co-administration of a Lonicera japonica extract and metformin (GEH+Met) in 4-week-old OLETF/LETO rats.

FIG. 22 indicates results showing the concentration changes of metformin in the blood over time (120, 240, 360, 380, 600, 720 min) at (a) 1 and 7 days or (b) 28 days after co-administration of a Lonicera japonica extract and metformin.

FIG. 23 is a result showing changes in metformin uptake when a Lonicera japonica extract and metformin are co-administered.

FIG. 24 is a result showing the viability of 3T3-L1 cells dependent upon administration of each Scutellaria baicalensis extract (HG: water extract, HG30: 30% ethanol extract, and HG100: 100% ethanol extract).

FIG. 25 is a result showing the viability of 3T3-L1 cells dependent upon co-administration of a Scutellaria baicalensis extract (HG: water extract, HG30: 30% ethanol extract or HG100: 100% ethanol extract) and metformin.

FIG. 26 is a result showing the viability of 3T3-L1 cells dependent upon administration of various concentrations (20, 50, 100, 200 μg/ml) of Scutellaria baicalensis extracts.

FIG. 27 is a result showing changes in intracellular ROS activity in HepG2 cells dependent upon co-administration of a Scutellaria baicalensis extract (HG: water extract, HG30: 30% ethanol extract or HG100: 100% ethanol extract) and metformin.

FIG. 28 is a result showing the inhibitory effects of administration of Scutellaria baicalensis extracts (HG: water extract, HG30: 30% ethanol extract, HG100: 100% ethanol extract) on nitrogen monoxide generation in RAW 264.7 cells.

FIG. 29 is a result showing the inhibitory effect of co-administration of a Scutellaria baicalensis extract (HG: water extract, HG30: 30% ethanol extract or HG100: 100% ethanol extract) and metformin on nitrogen monoxide generation in RAW 264.7 cells.

FIG. 30 is a result showing the suppressive effect of co-administration of a Scutellaria baicalensis extract (HG: water extract, HG30: 30% ethanol extract or HG100: 100% ethanol extract) and metformin on fat cell differentiation in 3T3-L1 cells.

FIG. 31 is a result showing an increased glucose uptake capacity of undifferentiated L6 rat myoblast cells by co-administration of a Scutellaria baicalensis extract (HG) and metformin.

FIG. 32 is a result showing changes in the protein expression level of PPAR-γ by co-administration of a Scutellaria baicalensis 100% ethanol extract (HG100) and metformin in 3T3-L1 cells.

FIG. 33 is a result showing changes in the protein expression level of PPAR-γ by co-administration of each of the Scutellaria baicalensis extracts at various concentrations (50, 100, 200 μg) and metformin in 3T3-L1 cells.

FIG. 34 is a result showing changes in the protein expression level of AMPK by co-administration of each of the Scutellaria baicalensis extracts at various concentrations (50, 100, 200 μg) and metformin in 3T3-L1 cells.

FIG. 35 is a result showing changes in the gene expression level of AMPK-α by administration of metformin (M), the combination of a Scutellaria baicalensis 30% ethanol extract and metformin (M-HGE) or the combination of a Scutellaria baicalensis water extract and metformin (M-HGW) in RAW 264.7 cells.

FIG. 36 is a result showing changes in the gene expression level of PPAR-α by administration of metformin (M), the combination of a Scutellaria baicalensis 30% ethanol extract and metformin (M-HGE) or the combination of a Scutellaria baicalensis water extract and metformin (M-HGW) in RAW 264.7 cells.

FIG. 37 is a result showing changes in the gene expression level of XBP-1 by administration of metformin (M), the combination of a Scutellaria baicalensis 30% ethanol extract and metformin (M-HGE) or the combination of a Scutellaria baicalensis water extract and metformin (M-HGW) in RAW 264.7 cells.

FIG. 38 is a result showing changes in the gene expression level of TNF-α by administration of metformin (M), the combination of a Scutellaria baicalensis 30% ethanol extract and metformin (M-HGE) or the combination of a Scutellaria baicalensis water extract and metformin (M-HGW) in RAW 264.7 cells.

FIG. 39 is a result showing changes in the gene expression level of IL-6 by administration of metformin (M), the combination of a Scutellaria baicalensis 30% ethanol extract and metformin (M-HGE) or the combination of a Scutellaria baicalensis water extract and metformin (M-HGW) in RAW 264.7 cells.

FIG. 40 indicates results showing (a) changes of insulin resistance and (b) changes in blood sugar level over time by co-administration of a Scutellaria baicalensis extract and metformin (HG+Met) in 4-week-old OLETF/LETO rats.

FIG. 41 indicates results showing the concentration changes of metformin in the blood over time (120, 240, 360, 380, 600, 720 min) at (a) 1 and 7 days or (b) 28 days after co-administration of a Scutellaria baicalensis extract and metformin.

FIG. 42 is a result showing changes in metformin uptake when a Scutellaria baicalensis extract and metformin are co-administered.

FIG. 43 is a result showing the viability of 3T3-L1 cells dependent upon administration of each Houttuynia cordata extract (OSC: water extract, OSC30: 30% ethanol extract, and OSC100: 100% ethanol extract).

FIG. 44 is a result showing the viability of 3T3-L1 cells dependent upon co-administration of a Houttuynia cordata extract (OSC: water extract, OSC30: 30% ethanol extract or OSC100: 100% ethanol extract) and metformin.

FIG. 45 is a result showing the viability of 3T3-L1 cells dependent upon administration of various concentrations (20, 50, 100, 200 μg/ml) of Houttuynia cordata extracts.

FIG. 46 is a result showing changes in intracellular ROS activity in HepG2 cells dependent upon co-administration of a Houttuynia cordata extract (OSC: water extract, OSC30: 30% ethanol extract or OSC100: 100% ethanol extract) and metformin.

FIG. 47 is a result showing the inhibitory effects of administration of Houttuynia cordata extracts (OSC: water extract, OSC30: 30% ethanol extract, OSC100: 100% ethanol extract) on nitrogen monoxide generation in RAW 264.7 cells.

FIG. 48 is a result showing the inhibitory effect of co-administration of a Houttuynia cordata extract (OSC: water extract, OSC30: 30% ethanol extract or OSC100: 100% ethanol extract) and metformin on nitrogen monoxide generation in RAW 264.7 cells.

FIG. 49 is a result showing the suppressive effect of co-administration of a Houttuynia cordata extract (OSC: water extract, OSC30: 30% ethanol extract or OSC100: 100% ethanol extract) and metformin on fat cell differentiation in 3T3-L1 cells.

FIG. 50 is a result showing an increased glucose uptake capacity of undifferentiated L6 rat myoblast cells by co-administration of a Houttuynia cordata extract (OSC: water extract, OSC30: 30% ethanol extract, or OSC100: 100% ethanol extract) and metformin.

FIG. 51 is a result showing an increased glucose uptake capacity of undifferentiated L6 rat myoblast cells by co-administration of a Houttuynia cordata 100% ethanol extract (OSC) and metformin.

FIG. 52 is a result showing an increased glucose uptake capacity of undifferentiated L6 rat myoblast cells by co-administration of each of the Houttuynia cordata extracts with various concentration (50, 100, and 200 μg/ml) and metformin.

FIG. 53 is a result showing the effect of co-administration of a Houttuynia cordata extract (OSC: water extract, OSC30: 30% ethanol extract, or OSC100: 100% ethanol extract) and metformin on the insulin secretion capacity of RIN-m5F insulinoma cells.

FIG. 54 is a result showing whether insulin resistance is improved by co-administration of a Houttuynia cordata extract and metformin (OSC+met1) in undifferentiated L6 rat myoblast cells.

FIG. 55 is a result showing changes in the expression level of dipeptidyl peptidase-4 (DPP-4) by co-administration of a Houttuynia cordata 100% ethanol extract (OSC100) and metformin in 3T3-L1 cells.

FIG. 56 is a result showing changes in the protein expression level of PPAR-γ by co-administration of a Houttuynia cordata 100% ethanol extract (OSC100) and metformin in 3T3-L1 cells.

FIG. 57 is a result showing changes in the protein expression level of PPAR-γ by co-administration of each of the Houttuynia cordata extracts at various concentrations (50, 100, 200 μg/ml) and metformin in 3T3-L1 cells.

FIG. 58 is a result showing changes in the protein expression level of AMPK by co-administration of each of the Houttuynia cordata extracts at various concentrations (50, 100, 200 μg) and metformin in 3T3-L1 cells.

FIG. 59 is a result showing changes in the gene expression level of AMPK-α by administration of metformin (M), the combination of a Houttuynia cordata 30% ethanol extract and metformin (M+USE) or the combination of a Houttuynia cordata water extract and metformin (M+USW) in RAW 264.7 cells.

FIG. 60 is a result showing changes in the gene expression level of PPAR-α by administration of metformin (M), the combination of a Houttuynia cordata 30% ethanol extract and metformin (M+USE) or the combination of a Houttuynia cordata water extract and metformin (M+USW) in RAW 264.7 cells.

FIG. 61 is a result showing changes in the gene expression level of PPAR-γ by administration of metformin (M), the combination of a Houttuynia cordata 30% ethanol extract and metformin (M+USE) or the combination of a Houttuynia cordata water extract and metformin (M+USW) in RAW 264.7 cells.

FIG. 62 is a result showing changes in the gene expression level of XBP-1 by administration of metformin (M), the combination of a Houttuynia cordata 30% ethanol extract and metformin (M+USE) or the combination of a Houttuynia cordata water extract and metformin (M+USW) in RAW 264.7 cells.

FIG. 63 indicates results showing (a) changes of insulin resistance and (b) changes in blood sugar level over time by co-administration of a Houttuynia cordata extract and metformin (OSC+Met) in 4-week-old OLETF/LETO rats.

FIG. 64 indicates results showing the concentration changes of metformin in the blood over time (120, 240, 360, 380, 600, 720 min) at (a) 1 and 7 days or (b) 28 days after co-administration of a Houttuynia cordata extract and metformin.

FIG. 65 is a result showing changes in metformin uptake when a Houttuynia cordata extract and metformin are co-administered.

### BEST MODE FOR CARRYING OUT THE INVENTION

In the present invention, it was confirmed that combined use of an extract extracted from any one selected from the group consisting of Lonicera japonica (Lonicerae Flos), Scutellaria baicalensis (Scutellariae Radix), and Houttuynia cordata (Houttuyniae Herba) and metformin increases the protein expression level of phosphorylated AMP-activated protein kinase (p-AMPK) and the gene expression levels of sirtuin 1 (SirT1), AMP-activated protein kinase-alpha (AMPK-α), peroxisome proliferator-activated receptor-alpha (PPAR-α), and peroxisome proliferator-activated receptor-gamma (PPAR-γ), which are associated with an anti-diabetic effect and an inhibitory effect on fat accumulation. In addition, it was confirmed that the combined use decreases the gene expression levels of X-box binding protein 1 (XBP-1), tumor necrosis factor-alpha (TNF-α), and interleukin-6 (IL-6), which are associated with the side effects of metformin. The present invention was completed on the basis thereof.

Hereinafter, the present invention will be described in detail.

It is an objective of the present invention to provide a pharmaceutical composition for improving an anti-diabetic effect, which is used in combination with metformin, an anti-diabetic drug, and includes an extract extracted from any one selected from the group consisting of Lonicera japonica (Lonicerae Flos), Scutellaria baicalensis (Scutellariae Radix), and Houttuynia cordata (Houttuyniae Herba).

In the present invention, the extracts may be extracted according to general methods of extracting extracts from natural products, which are known in the art, i.e., using general solvents under general temperature and pressure conditions. For example, in the present invention, a Houttuynia cordata extract may be extracted using, preferably ethanol, one or more solvents selected from the group consisting of water, alcohols having 1 to 4 carbons, and a combination thereof. In addition, extracts may be extracted from Houttuynia cordata using various methods such as hot water extraction, cold extraction, reflux extraction, and ultrasonic extraction, without being limited thereto.

The solvents may be removed from the prepared extracts by performing a filtration, concentration, or drying process or by performing all of filtration, concentration, and drying processes after finishing an extraction process. For example, the filtration process may be performed using a filter paper or a vacuum filter, the concentration process may be performed using a vacuum concentrator, and the drying process may be performed using a freeze-drying method and the like, without being limited thereto.

In addition, the extracts extracted by the solvents may be further subjected to a fractionation process using a solvent selected from the group consisting of hexane, methylene chloride, acetone, ethyl acetate, ethyl ether, chloroform, water and a mixture thereof. The fractionation may be performed at 4 to 120° C., but the present invention is not limited thereto.

The term “treatment” used in the present invention refers to all actions that improve the symptoms of diabetes mellitus or advantageously change the state of a diabetic patient by administration of a pharmaceutical composition according to the present invention.

“Diabetes mellitus”, a chronic metabolic disease that is an object to be prevented or treated by the composition of the present invention, can cause vascular disorders and malfunction of nerves, kidneys and retinas and the like over time, which may lead to loss of life. Diabetes mellitus, depending on generation mechanisms, is broadly divided into insulin-dependent diabetes mellitus (type 1 diabetes mellitus) and insulin-independent diabetes mellitus (type 2 diabetes mellitus), and in the present invention, diabetes mellitus preferably refers to insulin-independent diabetes mellitus. Generally, insulin-independent diabetes mellitus exhibits insulin resistance, and in an individual with diabetes mellitus, a high blood sugar level is maintained due to the failure of insulin action. Since chronic high blood sugar can cause cell death by damaging pancreatic beta-cells, effective regulation of blood sugar levels is needed when treating individuals with type 2 diabetes mellitus.

Gliclazide, glibenclamide, repaglinide, nateglinide, mitiglinide, rosiglitazone, pioglitazone, acarbose, voglibose and the like, preferably metformin, may be an anti-diabetic drug used in combination with the composition of the present invention, but the invention is not limited thereto.

For example, “metformin”, which is used as an anti-diabetic drug in the present invention, belongs to the biguanide class and has been used as a drug for primary treatment of patients with type 2 diabetes mellitus. However, use of metformin can cause side effects, such as loss of appetite, abdominal distension, nausea, diarrhea, and skin rashes, and thus special attention should be paid when using metformin.

Accordingly, to improve an anti-diabetic effect and decrease side effects of the anti-diabetic drug, the composition according to the present invention may be administrated simultaneously with or separately from the anti-diabetic drug, or the composition and the anti-diabetic drug may be administrated sequentially.

In addition, the composition according to the present invention may improve an anti-diabetic effect and at the same time, prevent or treat obesity.

“Obesity”, a disease that is an object to be prevented or treated by the composition of the present invention, refers to a condition in which excessive fat is accumulated in the body, which is attributed to proliferation and differentiation of fat cells due to metabolic disorders. When energy absorption is increased relative to energy consumption, the number and volume of fat cells are increased and consequently the mass of fat tissues is increased. Obesity at the cellular level refers to the increase in the number and volume of fat cells due to promotion of proliferation and differentiation of fat cells.

Obesity is closely associated with increase of insulin resistance, which is a major pathophysiological feature of type 2 diabetes mellitus. Insulin resistance, a condition in which blood sugar levels are not reduced despite a large amount of injected insulin, refers to a decrease in insulin sensitivity. It has been known that such decrease in insulin sensitivity is attributed to accumulation of fatty acids in beta-cells or insulin sensitive tissues such as the kidney, liver, and heart due to irregular secretion of adipokines and free fatty acids and consequent lipotoxicity.

In addition, the pharmaceutical composition according to the present invention may increase expression levels of one or more selected from the group consisting of phosphorylated AMP-activated protein kinase (p-AMPK) and genes encoding sirtuin 1 (SirT1), AMP-activated protein kinase-alpha (AMPK-α), peroxisome proliferator-activated receptor-alpha (PPAR-α), and peroxisome proliferator-activated receptor-gamma (PPAR-γ), respectively.

p-AMPK, SirT1, AMPK-α, PPAR-α and PPAR-γ, described above, are proteins that are associated with anti-diabetic effects and inhibitory effects on fat accumulation. AMP-activated protein kinase (AMPK) is activated when intracellular energy is deficient (i.e., when the amount of AMP is increased relative to the amount of ATP), and then the activated AMPK stimulates production of ATP, in which the synthesis of fatty acids, cholesterol, and the like is inhibited, whereas ATP is produced, i.e., the processes of fatty acid oxidation and glycolysis, resulting in restored normal energy balance. SirT1 has a deacetylase activity toward histone proteins and various transcription factors associated with cell growth, stress responses, endocrine regulation and the like. In addition, PPAR-α regulates the metabolism of glycolipids involved in lipolysis of neutral fat, and has a role in reducing triglyceride (TG) levels by activating lipoprotein lipase (LPL). PPAR-γ, one of transcriptional regulators in fat cells, has an important role in improving insulin sensitivity as well as a role in regulating the expression levels of enzymes responsible for differentiation of fat cells and fat synthesis/storage.

In addition, the composition according to the present invention may decrease expression levels of one or more selected from the group consisting of genes encoding X-box binding protein 1 (XBP-1), tumor necrosis factor-alpha (TNF-α), and interleukin-6 (IL-6), respectively.

The XBP-1, TNF-α and IL-6, described above, are proteins involved in the side effects of metformin. The XBP-1 is involved in endoplasmic reticulum stress, and the TNF-α and IL-6, as inflammatory cytokines involved in stimulating M2 macrophages, have roles in increasing inflammatory responses.

In one embodiment of the present invention, it was confirmed when a Lonicera japonica extract is administered alone or the Lonicera japonica extract and metformin are co-administered, cytotoxicity was not observed. In addition, it was experimentally confirmed that co-administration of the Lonicera japonica extract and metformin reduces intracellular reactive oxygen species, removes free radicals, inhibits nitrogen monoxide generation, and suppresses differentiation of fat cells (see Examples 1 to 5). In addition, it was confirmed that co-administration of the Lonicera japonica extract and metformin increases glucose uptake and insulin secretion and improves insulin resistance, and the co-administration inhibited the protein expression level of DPP-4 while increasing the protein expression levels of PPAR-γ, p-AMPK, and SirT1 (see Examples 6 to 9). In addition, it was confirmed that the co-administration improves an anti-diabetic effect and inhibits fat accumulation by increasing the gene expression levels of AMPK-α, PPAR-α, and PPAR-γ and decreases side effects caused by metformin by decreasing the gene expression levels of XBP-1, TNF-α, and IL-6. And it was confirmed, through in vivo animal experiments, that the co-administration decreases insulin resistance while not affecting the pharmacokinetic properties of metformin (see Examples 10 to 12).

In addition, in one embodiment of the present invention, it was confirmed when a Scutellaria baicalensis extract is administered alone or the Scutellaria baicalensis extract and metformin are co-administered, cytotoxicity was not observed. In addition, it was experimentally confirmed that co-administration of the Scutellaria baicalensis extract and metformin reduces intracellular reactive oxygen species, removes free radicals, inhibits nitrogen monoxide generation, and suppresses differentiation of fat cells (see Examples 13 to 17). In addition, it was confirmed that the co-administration increases glucose uptake and the protein expression levels of PPAR-γ and AMPK (see Examples 18 to 19). In addition, it was confirmed that the co-administration improves an anti-diabetic effect and inhibits fat accumulation by increasing the gene expression levels of AMPK-α and PPAR-α and decreases side effects caused by metformin by decreasing the gene expression levels of XBP-1, TNF-α, and IL-6. And it was confirmed, through in vivo animal experiments, that the co-administration decreases insulin resistance while not affecting the pharmacokinetic properties of metformin (see Examples 20 to 22).

In addition, in one embodiment of the present invention, it was confirmed when a Houttuynia cordata extract is administered alone or the Houttuynia cordata extract and metformin are co-administered, cytotoxicity was not observed. In addition, it was experimentally confirmed that co-administration of the Houttuynia cordata extract and metformin reduces intracellular reactive oxygen species, removes free radicals, inhibits nitrogen monoxide generation, and suppresses differentiation of fat cells (see Examples 23 to 27). In addition, it was confirmed that co-administration of the Houttuynia cordata extract and metformin increases glucose uptake and insulin secretion and improves insulin resistance, and the co-administration inhibited the protein expression level of DPP-4 while increasing the protein expression levels of PPAR-γ and AMPK (see Examples 28 to 31). In addition, it was confirmed that the co-administration improves an anti-diabetic effect and inhibits fat accumulation by increasing the gene expression levels of AMPK-α, PPAR-α, and PPAR-γ and decreases side effects caused by metformin by decreasing the gene expression level of XBP-1. In addition, it was confirmed, through in vivo animal experiments, that the co-administration decreases insulin resistance while not affecting the pharmacokinetic properties of metformin (see Examples 32 to 34).

The pharmaceutical composition according to the present invention may include a pharmaceutically acceptable carrier in addition to active ingredients. Pharmaceutically acceptable carriers, which are generally used in pharmaceutical preparations, include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil and the like, but the present invention is not limited thereto. In addition, the pharmaceutical composition may additionally include lubricants, wetting agents, sweeteners, flavoring agents, emulsifying agents, suspensions, preservatives and the like in addition to the carriers.

The pharmaceutical composition of the present invention may be administered orally or parenterally (for example, intravenous, subcutaneous, intraperitoneal or topical application) depending upon the desired method, and the dose, although varying depending on patient status and weight, degree of disease, drug type, route and time of administration, may be properly selected by those skilled in the art.

The pharmaceutical composition of the present invention is administered in a pharmaceutically effective dose. The term “pharmaceutically effective dose” according to the present invention refers to an amount sufficient to treat a disease at a reasonable benefit/risk ratio applicable to medical treatment, and the effective amount level may be determined by factors, including the disease type of a patient, severity, drug activity, sensitivity to a drug, administration time and route, emission rate, treatment period, and co-treated drugs, and other factors well known in medicine. The pharmaceutical composition according to the present invention may be administered as an individual therapeutic agent or in combination with other therapeutic agents, the composition may be administered sequentially or concurrently with conventional therapeutic agents, and the composition may be administered once or multiple times. Considering all of the above factors, it is important to administer a dose that can achieve the maximum effect in a minimal amount without side effects, which may be easily determined by those skilled in the art.

Specifically, the effective dose of the pharmaceutical composition of the present invention may be varied depending upon patient's age, sex, condition and body weight, the absorption degree of active ingredients in the body, the degree of inactivity, excretion rate, disease type, and co-treated drugs, and generally, 0.001 to 150 mg/kg body weight, preferably 0.01 to 100 mg, may be administered daily or every other day or one to three times a day. However, since the effective dose may be increased or decreased depending upon administration routes, the severity of obesity, sex, body weight, age and the like, the effective dose is not intended to limit the scope of the present invention in any way.

As another aspect of the present invention, the present invention provides a method of treating diabetes mellitus, which includes a step of administering the pharmaceutical composition to an individual. The term “individual” in the present invention refers to a subject who needs treatment for a disease, and more specifically, refers to humans or mammals such as non-human primates, mice, dogs, cats, horses and cattle.

Hereafter, the present invention will be described in more detail with reference to the following preferred examples. These examples are provided for illustrative purposes only and should not be construed as limiting the scope and spirit of the present invention.

### Example 1. Cytotoxicity Experiments for Lonicera japonica Extracts

100 μl of 3T3-L1 cells was aliquoted to each well of a 96-well plate at 3×10³ cells/well and incubated in a CO₂ incubator for 24 hours. Samples at various concentrations were added to each well and incubated for 24 hours, and thereafter 10 μl of EZ-Cytox was added to each well. After incubation for 2 hours in an incubator, the plate was shaken for 1 minute before measuring absorbance and then absorbance was measured at 450 nm using a 96-well plate reader. Cytotoxicity was measured according to extraction methods (water extract: GEH, 30% ethanol extract: GEH30, 100% ethanol extract: GEH100), whether metformin was co-administered, and concentration changes of Lonicera japonica extracts (20, 50, 100, 200 μg/ml).

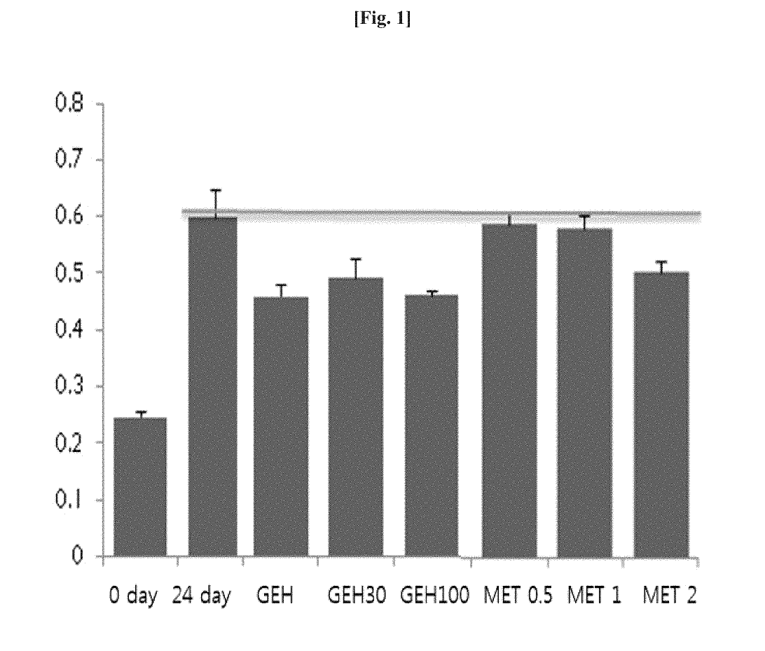

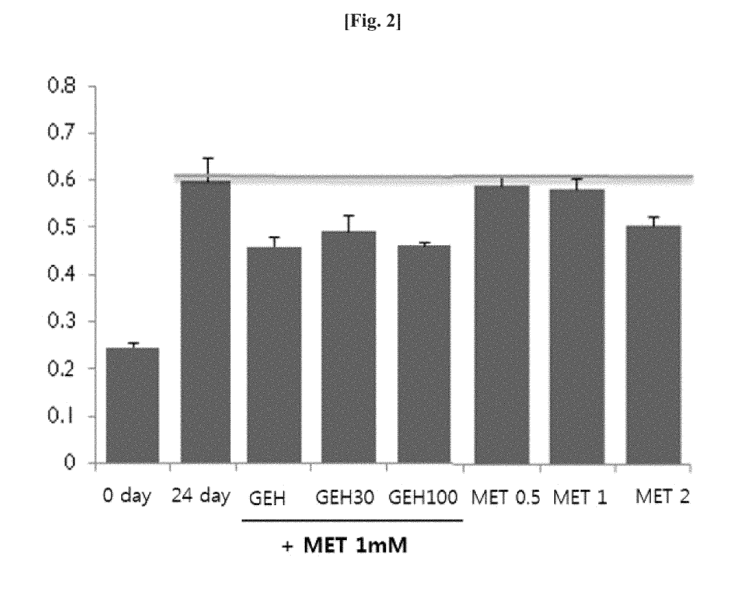

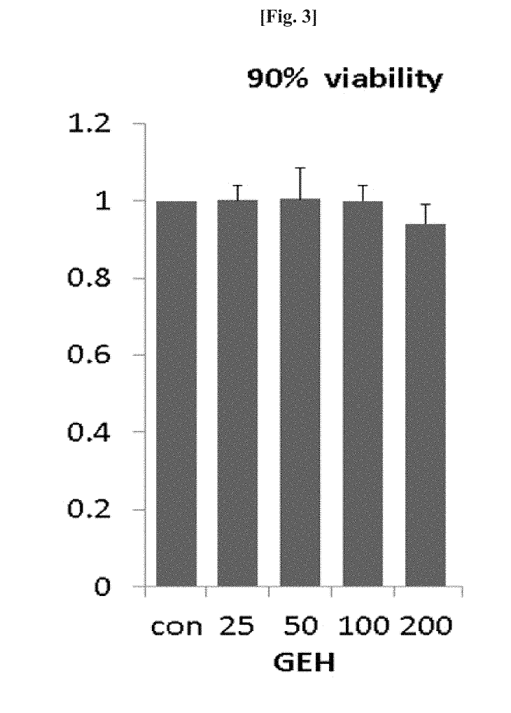

As a result, as illustrated in FIGS. 1 to 3, cytotoxicity was not observed in all groups regardless of extraction method and whether single administration or co-administration with metformin was carried out. In addition, despite an increase in the concentration of Lonicera japonica extracts administered, no cytotoxicity was observed.

### Example 2. Measurement of Changes in Intracellular Reactive Oxygen Species (ROS) Activity by Administration of Lonicera japonica Extracts

2 ml of HepG2 cells was aliquoted to each well of a 6-well plate at 3×10⁵ cells/well and incubated in a CO₂ incubator for 8 hours, and then the HepG2 cells were either treated with metformin alone or with metformin in combination with a Lonicera japonica extract and incubated for 6 hours, followed by cell harvesting. After centrifugation at 1200 g for 5 minutes, a supernatant was discarded, and the remaining HepG2 cells were treated with 5 μg/ml of DHR123, followed by incubation at 37° C. for 30 minutes. After additional centrifugation for 5 minutes, PBS washing was performed two times and filtration was performed. Intracellular reactive oxygen species activity was measured based on the value of fluorescence intensity obtained from FACS analysis.

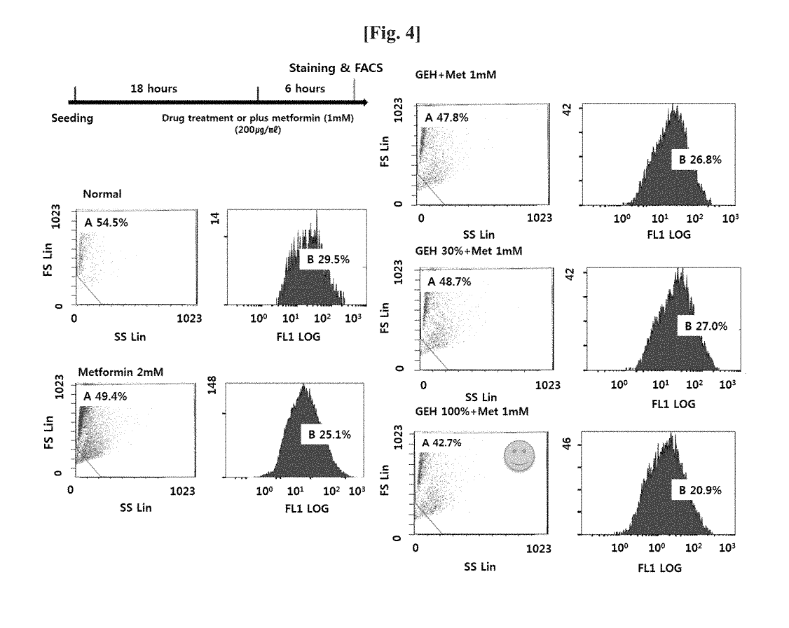

As a result, as illustrated in FIG. 4, a metformin-administered group (Metformin) exhibited a tendency of decreasing intracellular reactive oxygen species (ROS) activity compared to a normal group (Normal). In addition, co-administration of a Lonicera japonica extract and metformin further reduced intracellular ROS activity, and the most significant effect was observed in a Lonicera japonica 100% ethanol extract (GEH 100%+Met).

### Example 3. Measurement of DPPH Free Radical Scavenging Activity by Administration of Lonicera japonica Extracts

40 μl of a sample was mixed with 760 μl of 300 μM 2,2-diphenyl-1-picrylhydrazyl (DPPH) and the mixture was incubated at 37° C. for 30 minutes, and then the mixture was aliquoted to each well of a 96-well plate in triplicate and absorbance was measured at 515 nm using a microplate reader. BHT was used as a positive control group. In Example 3, depending upon 3 extraction methods (water, 30% ethanol, and 100% ethanol extractions), the DPPH free radical scavenging capacity of a Lonicera japonica extract was measured and IC₅₀ values were calculated.

As a result, BHT, a control group, showed a value of 113.85 μg/ml. In addition, when a Lonicera japonica water extract, a Lonicera japonica 30% ethanol extract, and a Lonicera japonica 100% ethanol extract were administered, as illustrated in the following Table 1, IC₅₀ values were 143.36 μg/ml, 154.35 μg/ml, and 146.93 μg/ml, respectively, demonstrating that these extracts have an excellent free radical scavenging capacity. The most significant effect was observed in a Lonicera japonica water extract (Water extract).

TABLE 1

| IC₅₀ | Lonicera japonica |
|---|---|
| Water extract | 143.36 μg/μl |
| 30% EtoH | 154.35 μg/μl |
| 100% EtoH | 146.93 μg/μl |

### Example 4. Measurement of Capacity of Lonicera japonica Extracts for Inhibiting Nitrogen Monoxide Generation

To compare an anti-inflammatory function, an in vitro model of LPS-induced nitrogen monoxide (NO) generation was used in an experiment, and NO measurement was carried out using a cell supernatant based on the GRIESS reaction (Green et al., 1982). RAW 264.7 cells were seeded at a density of 1.5×10⁵ cells/ml and pre-treated with samples diluted at various concentrations, and after 1 hour, the pretreated cells were treated with 1 μg/ml of lipopolysaccharide (LPS: Sigma, St Louis, Mo., USA), followed by incubation for 24 hours. 50 μl of a cell culture supernatant and 50 μl of 1% (w/v) sulfanilamide, a GRIESS reagent, were added to each well of a 96-well plate, and the 96-well plate shaded from light was incubated at room temperature for 10 minutes and then 50 μl of 0.1% (w/v) N-1-naphthylethylenediamine dissolved in 2.5% (v/v) phosphoric acid was added to each well of the 96-well plate and mixed, followed by incubation under dark conditions for 10 minutes. Absorbance was read at 540 nm using a microplate reader (Molecular Devices, CA, USA) within 30 minutes after finishing incubation. NO production was calculated using a nitric oxide standard solution.

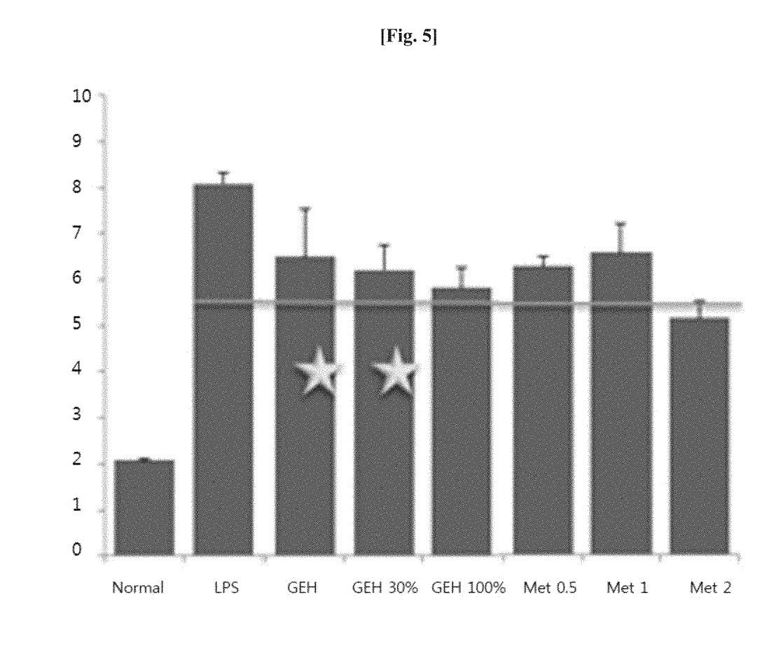

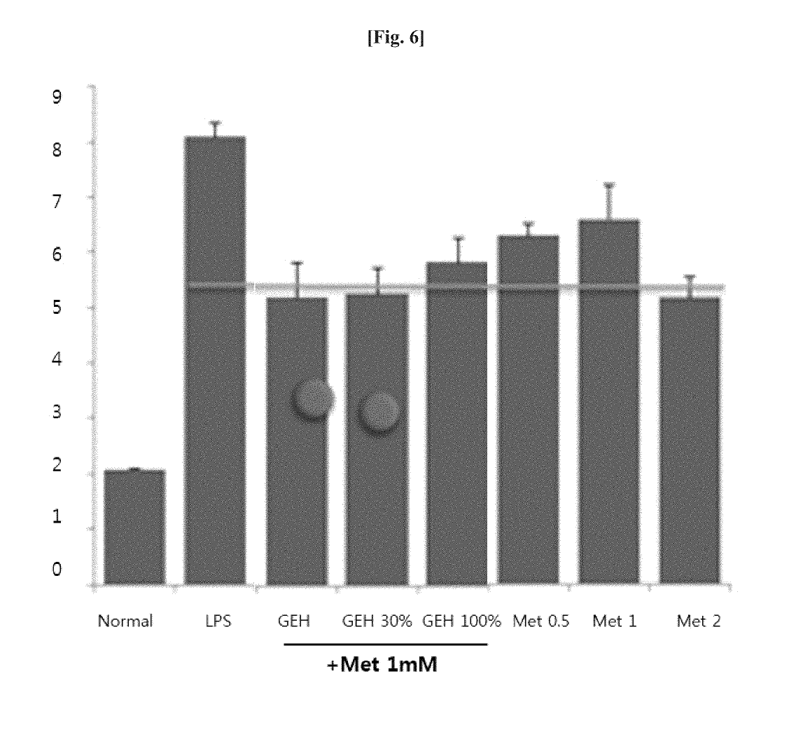

As a result, as illustrated in FIG. 5, the production amount of nitrogen monoxide was decreased in groups administered with metformin alone (Met 0.5, Met 1, Met 2) compared to an LPS-administered group (LPS). When a Lonicera japonica extract was administered alone, the production amount of nitrogen monoxide was also decreased regardless of extraction method compared to the LPS-administered group (LPS). In addition, as illustrated in FIG. 6, it was confirmed that the production amount of nitrogen monoxide was further decreased in groups co-administered with metformin and a Lonicera japonica extract compared to groups administered with metformin alone.

### Example 5. Confirmation of Inhibitory Effects of Lonicera japonica Extracts on Fat Cell Differentiation

After seeding 3T3-L1 cells into a 6-well plate at a density of 5×10⁵ cells/well, the cells were cultured until reaching full confluence. The pre-existing culture medium for the cells was exchanged with DMEM (differentiation media) containing 1 μM dexamethasone, 0.5 mM IBMX, and 10 μg/ml insulin, and the cells were cultured for 48 hours and then were treated with DMEM (maturation media) containing 10 μg/ml insulin to induce differentiation. Differentiation-induced fat cells produced as a result of the process were treated with samples at various concentrations or a positive control group, and inhibitory effects on fat cell differentiation were analyzed using an Oil red O staining method, TG, and a TC assay.

As a result, as illustrated in FIG. 7, a group administered with metformin alone (Met) exhibited a tendency of decreasing lipid formation attributed to differentiation of 3T3-L1 cells, preadipocytes, compared to a control group. In addition, groups co-administered with metformin and a Lonicera japonica extract exhibited an inhibition effect far superior to that of the group administered with metformin alone, and a Lonicera japonica 30% ethanol extract (GEH 30%+Met) showed the most significant effect.

### Example 6. Glucose Uptake Assay Depending Upon Administration of Lonicera japonica Extracts

Undifferentiated L6 rat myoblast cells were differentiated into myotube cells using 2% horse serum. As another method, HepG2 cells were seeded into each well of a 96-well back/clear bottom plate and incubated, and then the pre-existing medium was exchanged with a glucose free medium to provide a glucose starvation condition to the cells, followed by incubation for 12 hours. Thereafter, a medium of the cell culture was exchanged with a glucose free medium containing various samples and 2-NBDG, a fluorescent reagent, at a concentration of 100 μg/ml and then incubated for 6 to 12 hours. After incubation, the cell culture was washed two times with DPBS and then subjected to measurement of fluorescence intensity at 485/535 nm (excitation/emission=485/535 nm) using a fluorescence microplate reader. When performing measurement, apigenin, a compound that inhibits glucose uptake, was used as a control.

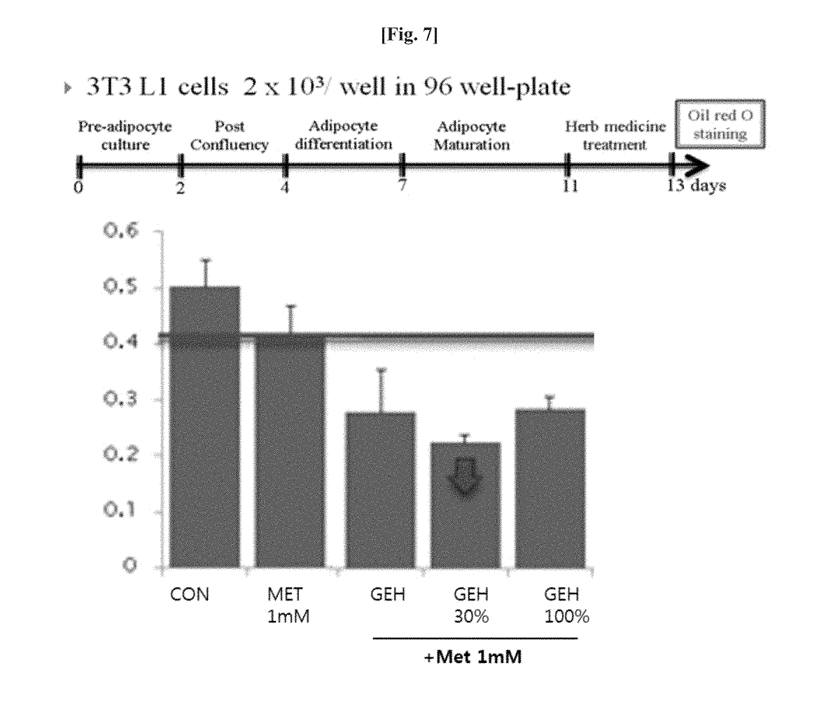

As a result, as illustrated in FIG. 8, a group administered with metformin alone (Met) exhibited an increased capacity of glucose uptake compared to a control group. When comparing the group administered with metformin alone, a group co-administered with a Lonicera japonica 100% ethanol extract and metformin exhibited a significant increase in the capacity of glucose uptake.

### Example 7. Insulin Secretion Assay Depending Upon Administration of Lonicera japonica Extracts

RIN-m5F insulinoma cells were cultured in a RPMI 1640 medium (WELGENE Inc., Korea) containing 10% FBS, 0.6% penicillin streptomycin (PS), and 300 mg/l L-glutamine in a CO₂ incubator set to 37° C. with 5% CO₂. RIN-m5F cells were aliquoted to each well of a 12-well plate at 3×10⁵ cells/well and incubated for 3 days, and then the cells were treated with the combination of 0.75 mM metformin and each of the Lonicera japonica extracts (GEH, GEH 30, or GEH 100). After culturing for 2 days, the pre-existing medium was discarded from the cells, and the cell culture was washed two times with modified Krebs-Ringer Bicarbonate Buffer (KRBB-HEPES, 134 mmol/1 NaCl, 4.8 mmol/1 KCl, 1 mmol/1 CaCl₂, 1.2 mmol/1 MgSO₄, 1.2 mmol/1 KH₂PO₄, 5 mmol/1 NaHCO₃, 10 mmol/1 HEPES, 1 mg/ml BSA, pH 7.4) and incubated in a KRBB-HEPES buffer containing 20 mM glucose for 1 hour. A portion of cell culture supernatant was subjected to centrifugation at 4° C. for 10 minutes, and after centrifugation, a supernatant was collected and stored at −20° C. for further use. The amount of secreted insulin was measured using a rat insulin ELISA kit (Mercodia, Sweden), and an insulin secretion amount per gram of proteins was calculated by measuring the concentration of cellular proteins in each well.

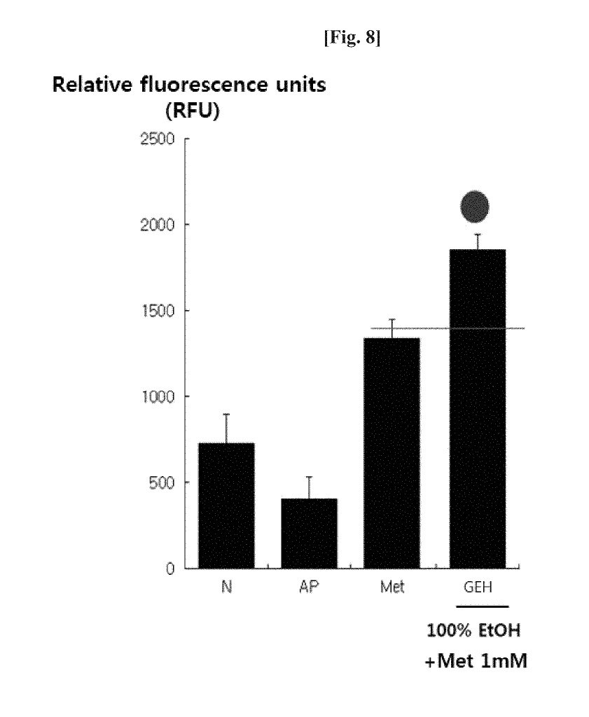

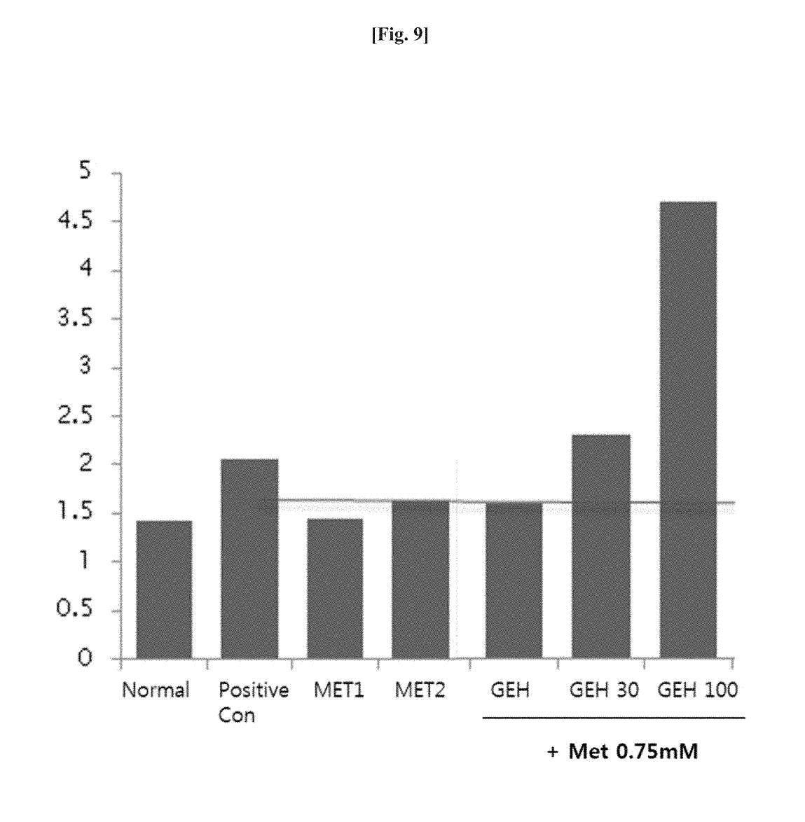

As a result, as illustrated in FIG. 9, while significant changes in the amounts of insulin secretion were not observed in groups administered with metformin alone (MET1, MET2), significant changes in the amounts of insulin secretion were observed in groups co-administered with metformin and each Lonicera japonica extract. The group co-administrated with metformin and a Lonicera japonica 100% ethanol extract exhibited the most significant change.

### Example 8. Insulin Resistance Assay Depending Upon Administration of Lonicera japonica Extracts

Undifferentiated L6 rat myoblast cells were seeded into each well of a 96-well back/clear bottom plate and differentiated into myotube cells by adding 2% horse serum and then subjected to measurement of fluorescence intensity. The pre-existing medium of the differentiated L6 cells was exchanged with a glucose free medium to provide a glucose starvation condition to the cells, followed by incubation for 2 hours. After the incubation period, the cells under the glucose starvation condition were treated with samples at various concentrations. Thereafter, a medium of the cell culture was exchanged with a glucose free medium containing 5 mM glucosamine and incubated for 6 to 12 hours to induce insulin resistance. After removing a supernatant from the cell culture, the cell culture was treated with a glucose-free medium containing 100 μg/ml 2-NBDG and subsequently incubated for 6 hours, and then the cell culture was washed two times with DPBS and subjected to measurement of fluorescence intensity at 485/535 nm (excitation/emission=485/535 nm) using a fluorescence microplate reader.

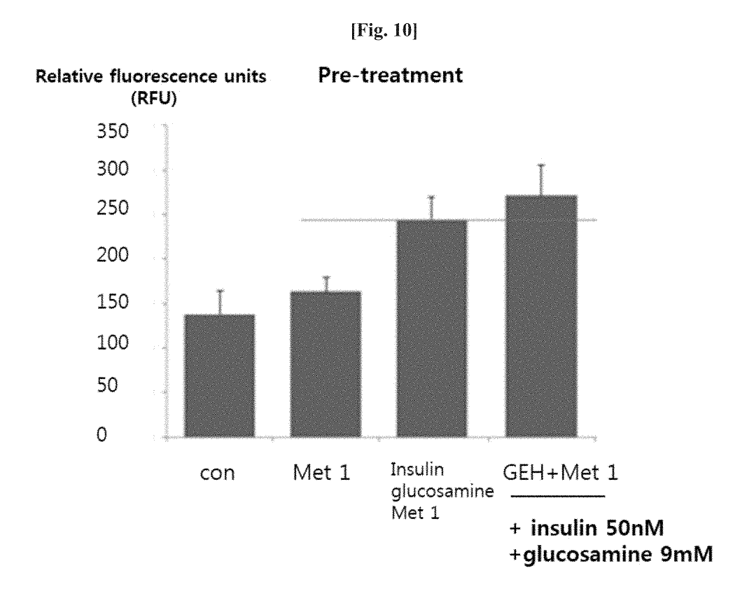

As a result, as illustrated in FIG. 10, under a condition of treating with insulin and glucosamine, a group co-administered with a Lonicera japonica extract and metformin exhibited superior glucose uptake compared to a group administered with metformin alone, indicating that a synergistic effect on improving insulin resistance can be obtained when using a combination of metformin and a Lonicera japonica extract.

### Example 9. Confirmation of Changes in Expression Levels of Related Proteins Depending Upon Administration of Lonicera japonica Extracts

### 9-1. DPP-4 and PPAR-γ Protein Expression

3T3-L1 preadipocytes were cultured in DMEM (WELGENE Inc., Korea) containing 10% FBS and 1% penicillin streptomycin (PS) in a CO₂ incubator set to 37° C. with 5% CO₂. The cells were aliquoted to each well of a 6-well plate for cell culture at 8×10⁴ cells/well. To induce cell differentiation, the cells were cultured until reaching 50 to 60% confluence and subsequently, the pre-existing medium was exchanged with a differentiation-inducing DMEM medium containing 0.5 mM IBMX, 1 μM dexamethasone, 10 μg/ml insulin and 10% FBS, and then the cells were cultured for 3 days. After 3 days, the medium of the cell culture was exchanged with a DMEM medium containing 10 μg/ml insulin and 10% FBS and the cell culture was cultured while exchanging the medium every 2 days. At 5 days after differentiation, the cell culture was treated with samples and incubated for 24 hours. The cells in the 6-well plate were washed two times with PBS and subjected to lysis using a RIPA buffer (50 mM Tris-HCl pH 7.4, 1% NP-40, 0.25% sodium deoxycholate, 150 mM NaCl, 1 mM EDTA, 1 mM PMSF, 1 mM NaF, 1 mM sodium, 1 μg/ml aprotinin, leupeptin, pepstatin), and then the lysate was subjected to centrifugation at 12,000 rpm for 20 minutes to obtain a supernatant containing proteins. After performing quantification according to the BCA (Thermo Scientific, USA) method, electrophoresis was carried out on a 10% polyacrylamide gel. After electrophoresis, proteins on the gel were transferred to a PVDF membrane at 200 mA for 90 minutes, and the membrane was treated with a blocking buffer containing 5% skim milk or 5% BSA to reduce background signals due to non-specific proteins and incubated with primary antibodies at 4° C. overnight, and then the membrane was washed three times with TBS-T, in which each washing was performed for 10 minutes. Thereafter, the membrane was treated with secondary antibodies at room temperature for 1 hour and then washed three times with TBS-T, in which each washing was performed for 10 minutes, and the membrane was treated with an ECL (NEURONEX, Korea) solution and subsequently subjected to measurement of protein expression levels using LAS-3000 (FUJIFILM, Japan).

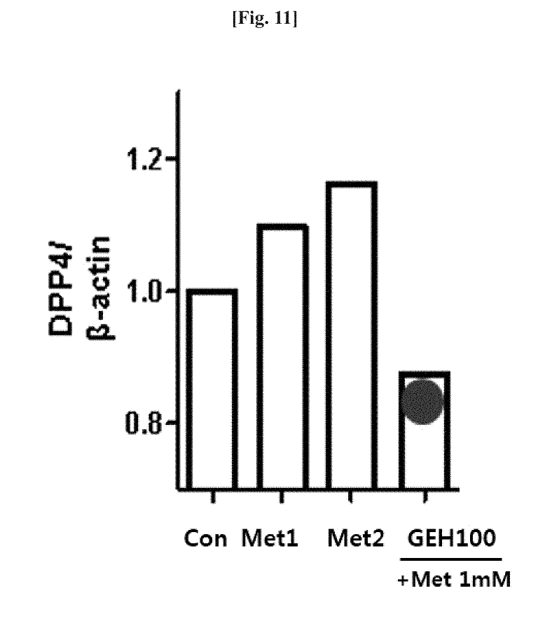

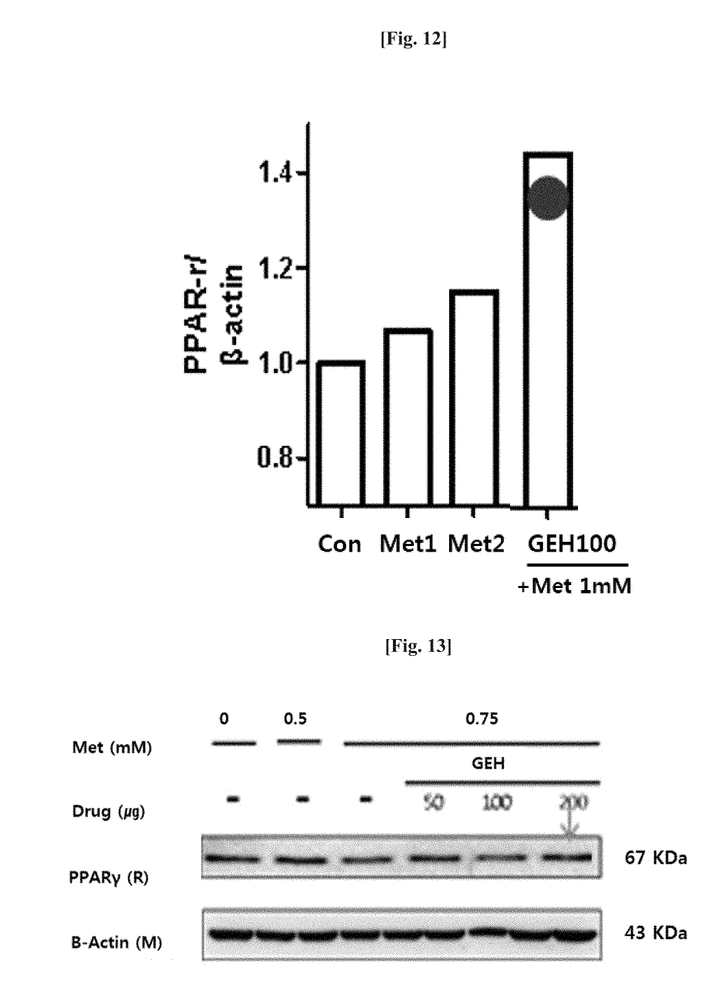

As a result, as illustrated in FIGS. 11 to 13, when compared with groups administered with metformin alone (MET1 and MET2), the expression level of dipeptidyl peptidase-4 (DPP-4) was decreased in a group co-administered with metformin and a Lonicera japonica extract, whereas the expression level of PPAR-γ was significantly increased in the same. In Example 9, inhibition of the expression of DPP-4, an enzyme responsible for degrading incretin, leads to stimulation of synthesis/secretion of insulin, inhibition of glucagon secretion and inhibition of glucose synthesis in the liver, and thus blood sugar levels can be controlled by regulating the expression of DPP-4. It has been known that increasing PPAR-γ expression has a positive effect on increasing insulin sensitivity. Accordingly, the results indicate that co-administration of metformin and a Lonicera japonica extract can further improve an anti-diabetic effect.

9-2. p-AMPK and Sirt1 Protein Expression

RAW 264.7 cells, a macrophage cell line, were obtained from the Korean Cell Line Bank (KCLB, Seoul, Korea), and DMEM containing 10% FBS, 2 mM L-glutamine, 100 U/ml penicillin, and 100 μg/ml streptomycin was used as a medium for culturing the RAW 264.7 cells. The cells were cultured in a CO₂ incubator set to 37° C. with 5% CO₂ and 95% O₂. Lonicera japonica extracts (100% water and 30% ethanol extracts) used in the experiments of the present invention were provided from the College of Pharmacy, Dongguk University. Experiments were performed for a total of 4 groups, including a normal group (N), a metformin-administered group (M), a group administered with a Lonicera japonica extract (30% ethanol extract) (GEH), and a group administered with metformin and a Lonicera japonica extract (30% ethanol extract) (M+GEH).

DMEM containing 10% FBS, 2 mM L-glutamine, 100 U/ml penicillin, and 100 μg/ml streptomycin was used as a medium for culturing the RAW 264.7 cells, and the cells were cultured under conditions of 37° C., 5% CO₂, and 90% humidity. The cultured cells were maintained while exchanging the culture medium once every 2 to 3 days. When the cells were fully differentiated, the cell culture was washed with phosphate buffered saline (PBS) and then the cells were detached from a culture dish using a trypsin-EDTA solution. The separated cells were subjected to centrifugation to collect the same, and then the collected cells were mixed with a fresh medium and subcultured.

To prepare a cell lysate, cells treated with the composition according to the present invention were washed with a 10 mM phosphate buffer (pH 7.4) solution containing 150 mM NaCl (in PBS) and subjected to lysis with a PBS solution containing 0.1% SDS and 10 mM β-mercaptoethanol. After cell harvesting, a cell lysate was loaded onto an 8% SDS-polyacrylamide gel and subjected to electrophoresis. The protein bands existing on the gel were transferred to a nitrocellulose membrane (Schleicher and Schull, Dassel, Germany) using a semi-dry blotter (MilliBlot-SDE system, Millipore, Bedford, Mass., USA). The membrane was washed one time with a 10 mM Tris-buffered saline buffer (TBS, pH 7.2) containing 0.1% Tween-20 (TBS-T) and then soaked in a Tris-buffered saline buffer (TBS, pH 7.2) containing 3% skim milk and incubated at room temperature for 1 hour for blocking reaction. The membrane was incubated with anti-Sift1 antibodies, anti-p-AMPK antibodies, anti-AMPK antibodies (Cell Signaling Technology, DV, USA) or anti-beta actin antibodies. After incubation for 2 hours, the membrane was incubated with horseradish peroxidase-conjugated goat anti-Rabbit IgG antibodies (Santa Cruz Biotechnology, CA, USA) (diluted 1:1000) as a secondary antibody. Thereafter, the membrane was treated with an Enhanced Chemiluminescence (ECL) solution (Amersham Corp., Newark, N.J., USA) and subsequently analyzed using an image reader (LAS-3000, Fuji Photo Film, Tokyo, Japan). The intensities of the protein bands were measured using densitometry, and protein quantification was analyzed based on beta-actin.

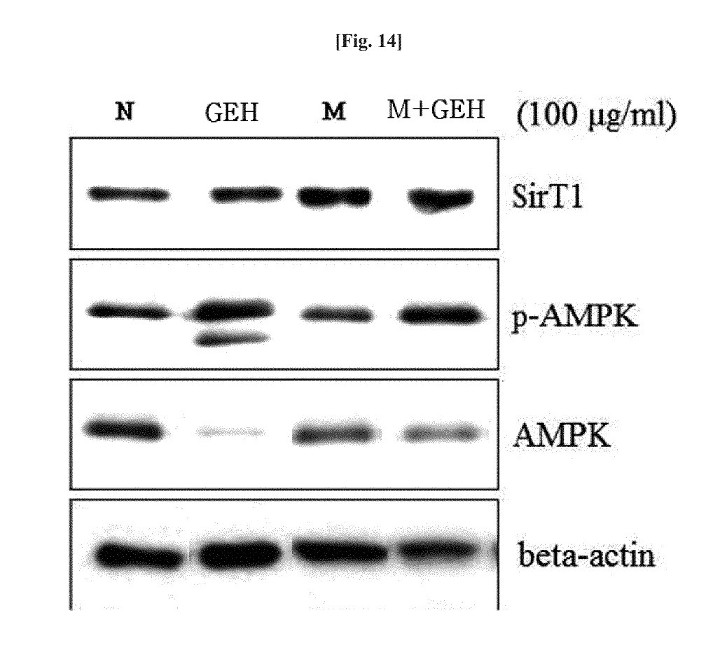

As a result, as illustrated in FIG. 14, the expression levels of Sirt1 and p-AMPK proteins were significantly increased in a group co-administered with metformin and a Lonicera japonica extract (30% ethanol extract) (M+GEH) compared to a group administered with a Lonicera japonica extract (30% ethanol extract) alone (GEH) and a group administered with metformin alone (M).

### Example 10. Confirmation of Changes in Expression Levels of Related Genes Depending Upon Administration of Lonicera japonica Extracts

10-1. Expression of Genes Associated with Anti-Diabetic Effect

To confirm whether combined use of metformin and a Lonicera japonica extract affects the expression of genes associated with an anti-diabetic effect, the gene expression levels of AMPK-α, PPAR-α, and PPAR-γ of RAW 264.7 cells administered with metformin alone (M) and RAW 264.7 cells administered with metformin and a Lonicera japonica extract (30% ethanol or 100% water) were compared using real-time PCR. RAW 264.7 cells were harvested according to the same method as described in Example 9-2. Experiments were performed for a total of 4 groups, including a normal group (N), a metformin-administered group (M), a group administered with metformin and a Lonicera japonica extract (30% ethanol) (M+GEH) and a group administered with metformin and a Lonicera japonica extract (water extract) (M+GEHW).

Total RNA was separated and purified using TRIsure (Bioline, USA) according to a protocol. 1 μg of total RNAs was subjected to a reverse transcription reaction using a cDNA synthesis kit (Sprint™RT Complete Oligo-(dT)18, Clontech, Mountain View, Calif., USA) according to a protocol for synthesizing first strand cDNA. The produced RT-PCR sample was subjected to real-time PCR reaction, in which the final reaction volume was adjusted to 20 μl and Light Cycler-Fast Start DNA Master SYBR Green (Roche Applied Science, Indianapolis, Ind., USA) and a Light Cycler instrument (Roche Applied Science) were used.

DNA sequences of primers used in Example 10-1 are as follows.

TABLE 2

| Genus | | | |
|---|---|---|---|
| Specific | | | Annealing |
| Primers | Direction | Sequence | temp. |
| Beta-actin | F | 5′-GCAAGTGCTTCTAGGCGGAC-3′ | 52° C. |
| | | (SEQ ID NO. 1) | |
| | R | 5′-AAGAAAGGGTGTAAAACGCAGC-3′ | |
| | | (SEQ ID NO. 2) | |
| AMPK alpha 1 | F | 5′-AAGCCGACCCAATGACATCA-3′ | 49° C. |
| | | (SEQ ID NO. 3) | |
| | R | 5′-CTTCCTTCGTACACGCAAAT-3′ | |
| | | (SEQ ID NO. 4) | |
| PPAR-alpha | F | 5′-GCCTGTCTGTCGGGATGT-3′ | 50° C. |
| | | (SEQ ID NO. 5) | |
| | R | 5′-GGCTTCGTGGATTCTCTTG-3′ | |
| | | (SEQ ID NO. 6) | |
| PPAR-gamma | F | 5′-GCCCTTTGGTGACTTTATGGA-3′ | 51° C. |
| | | (SEQ ID NO. 7) | |
| | R | 5′-GCAGCAGGTTGTCTTGGATG-3′ | |
| | | (SEQ ID NO. 8) | |

PCR amplification was performed according to PCR steps, consisting of a pre-incubation step at 95° C. for 10 minutes and 35 (for beta-actin) or 45 (for C/EBPa) cycles of amplification (denaturation at 95° C. for 10 seconds, annealing at 52° C. for 10 seconds, and extension at 72° C. for 15 seconds). Total RNA was separated and purified using TRIsure (Bioline, USA) according to a protocol. 1 μg of total RNAs was subjected to a reverse transcription reaction using a cDNA synthesis kit (Sprint™RT Complete Oligo-(dT)18, Clontech, Mountain View, Calif., USA) according to a protocol for synthesizing first strand cDNA. The produced RT-PCR sample was subjected to real-time PCR reaction, in which the final reaction volume was adjusted to 20 μl and Light Cycler-Fast Start DNA Master SYBR Green (Roche Applied Science, Indianapolis, Ind., USA) and a Light Cycler instrument (Roche Applied Science) were used.

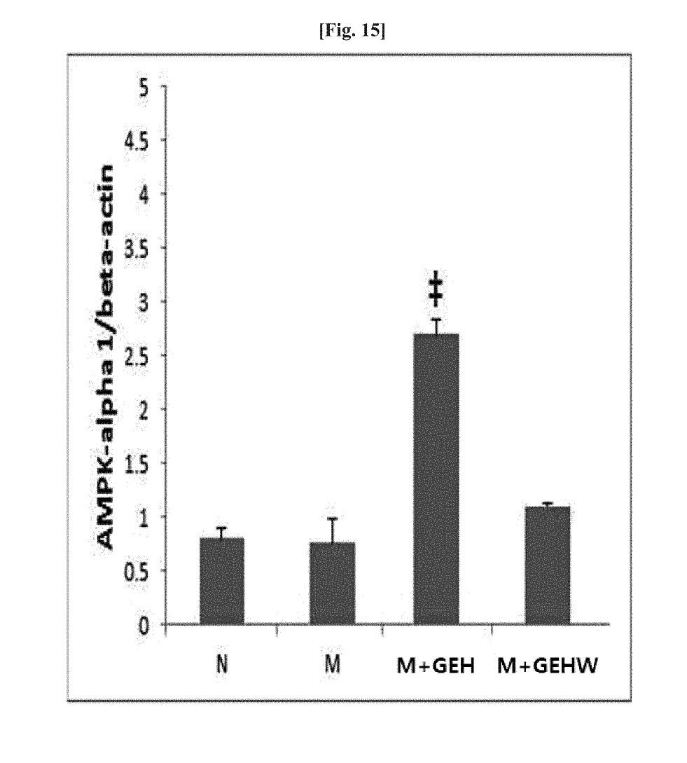

As a result, as illustrated in FIG. 15, a normal group (N) and a metformin-administered group (M) showed 0.80 and 0.76 for the gene expression levels of AMPK-α, respectively. A group co-administered with metformin and a Lonicera japonica extract (30% ethanol) (M+GEH) and a group co-administered with metformin and a Lonicera japonica extract (100% water) (M+GEHW) exhibited increased gene expression of AMPK-α. In particular, the M+GEH group exhibited a significant increase in AMPK-α gene expression, showing a value of 2.70.

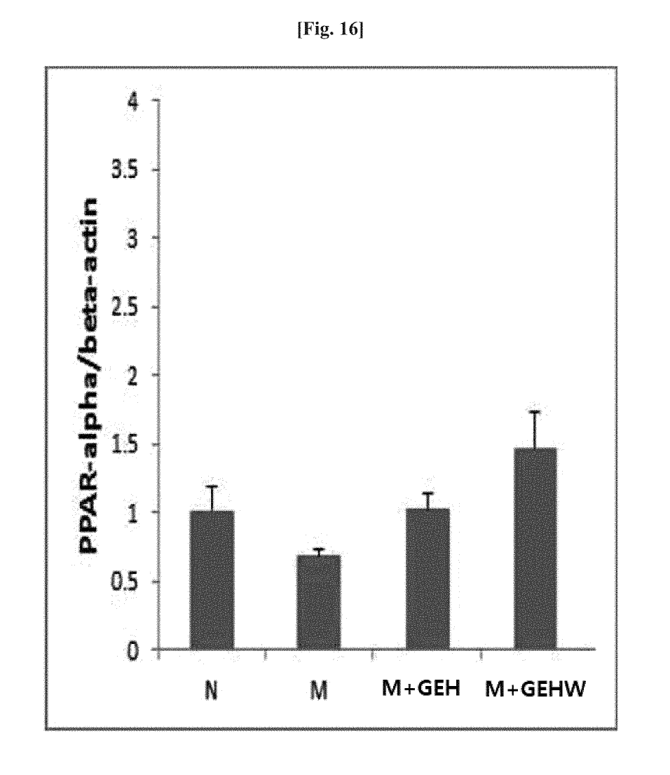

In addition, as illustrated in FIG. 16, a normal group (N) and a metformin-administered group (M) exhibited 1.01 and 0.68 for the gene expression levels of PPAR-α, respectively. A group co-administered with metformin and a Lonicera japonica extract (30% ethanol) (M+GEH) and a group co-administered with metformin and a Lonicera japonica extract (100% water) (M+GEHW) exhibited increased gene expression of PPAR-α.

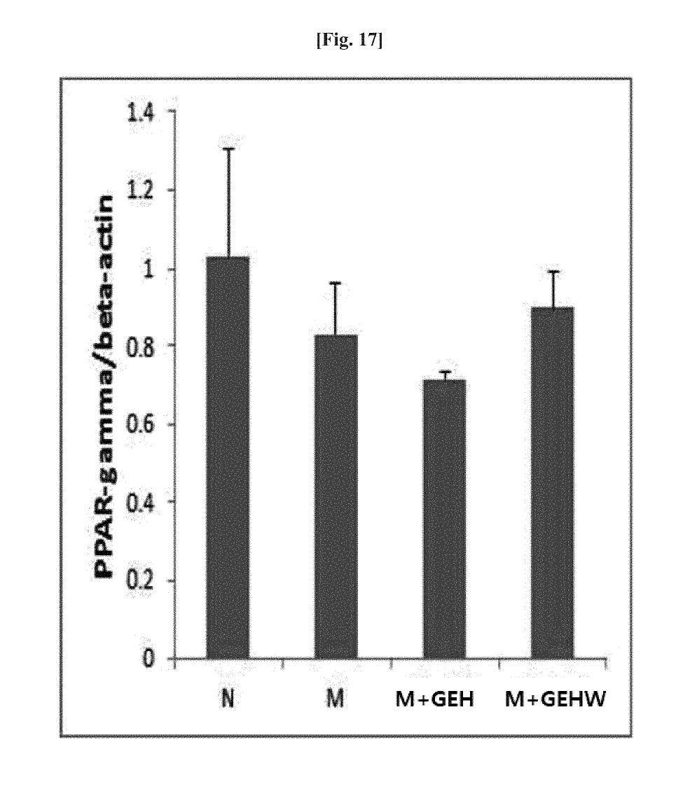

In addition, as illustrated in FIG. 17, a normal group (N) and a metformin-administered group (M) exhibited 1.03 and 0.83 for the gene expression levels of PPAR-γ, respectively. A group co-administered with metformin and a Lonicera japonica extract (100% water) (M+GEHW) exhibited increased gene expression of PPAR-γ, showing a value of 0.90.

10-2. Expression of Genes Associated with Side Effects of Metformin

To identify the effect of co-administration of metformin and a Lonicera japonica extract on expression of genes, which are associated with side effects caused by metformin, the gene expression levels of XBP-1, TNF-α, and IL-6 of RAW 264.7 cells administered with metformin alone (M) and RAW 264.7 cells administered with metformin and a Lonicera japonica extract (30% ethanol or 100% water) were compared using real-time PCR. RAW 264.7 cells were harvested according to the same method as described in Example 9-2. Experiments were performed for a total of 4 groups, including a normal group (N), a metformin-administered group (M), a group administered with metformin and a Lonicera japonica extract (30% ethanol) (M+GEH) and a group administered with metformin and a Lonicera japonica extract (water extract) (M+GEHW).

To identify the gene expression levels of XBP-1, TNF-α, and IL-6, real-time PCR was performed according to the same method as described in Example 10-1 except primers.

DNA sequences of primers used in Example 10-2 are as follows.

TABLE 3

| Genus | | | |
|---|---|---|---|
| Specific | | | Annealing |
| Primers | Direction | Sequence | temp. |
| beta-actin | F | 5′-GCAAGTGCTTCTAGGCGGAC-3′ | 52° C. |
| | | (SEQ ID NO. 1) | |
| | R | 5′-AAGAAAGGGTGTAAAACGCAGC-3′ | |
| | | (SEQ ID NO. 2) | |
| XBP-1 | F | 5′-TGGCCGGGTCTGCTGAGTCCG-3′ | 51° C. |
| | | (SEQ ID NO. 9) | |
| | R | 5′-GTCCATGGGAAGATGTTCTGG-3′ | |
| | | (SEQ ID NO. 10) | |
| TNF-alpha | F | 5′-GAACTGGCAGAAGAGGCACT-3′ | 52° C. |
| | | (SEQ ID NO. 11) | |
| | R | 5′-AGGGTCTGGGCCATAGAACT-3′ | |
| | | (SEQ ID NO. 12) | |
| IL-6 | F | 5′-AGTTGCCTTCTTGGGACTGA-3′ | 49° C. |
| | | (SEQ ID NO. 13) | |
| | R | 5′-CAGAATTGCCATTGCACAAC-3′ | |
| | | (SEQ ID NO. 14) | |

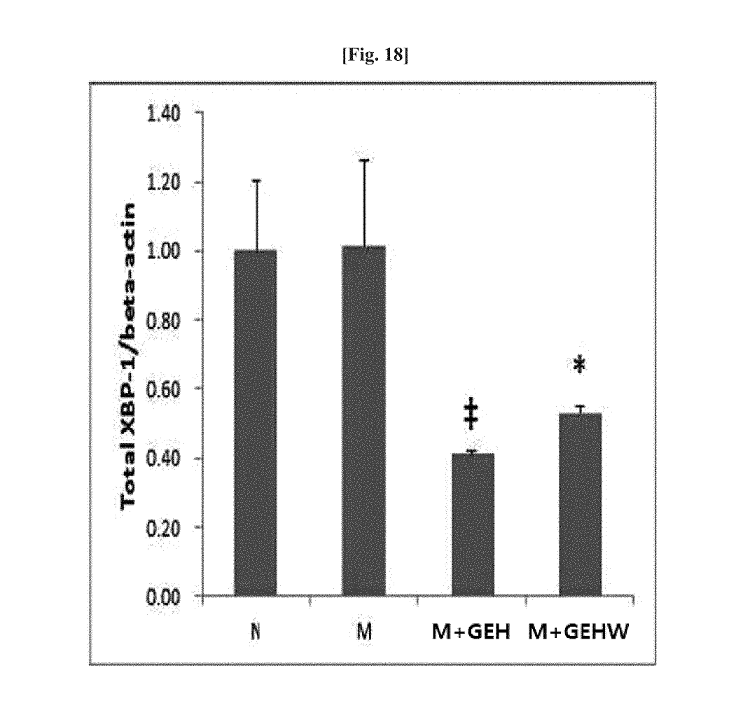

As a result, as illustrated in FIG. 18, a normal group (N) and a metformin-administered group (M) exhibited 1.00 and 1.01 for the gene expression levels of XBP-1, respectively. A group co-administered with metformin and a Lonicera japonica extract (30% ethanol) (M+GEH) and a group co-administered with metformin and a Lonicera japonica extract (100% water) (M+GEHW) exhibited decreased gene expression of XBP-1, showing values of 0.41 and 0.53, respectively.

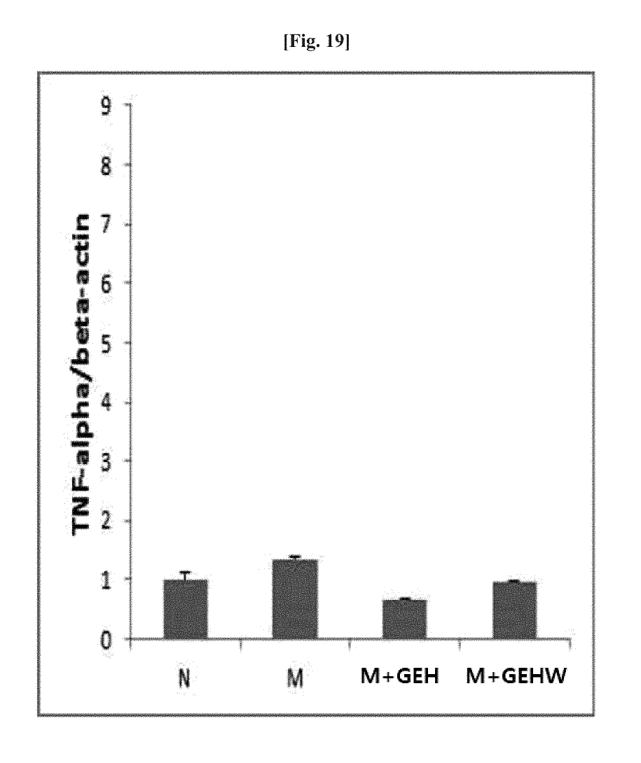

In addition, as illustrated in FIG. 19, a normal group (N) and a metformin-administered group (M) exhibited 1.01 and 1.34 for the gene expression levels of TNF-α, respectively. A group co-administered with metformin and a Lonicera japonica extract (30% ethanol) (M+GEH) and a group co-administered with metformin and a Lonicera japonica extract (100% water) (M+GEHW) exhibited decreased gene expression of TNF-α, showing values of 0.66 and 0.97, respectively.

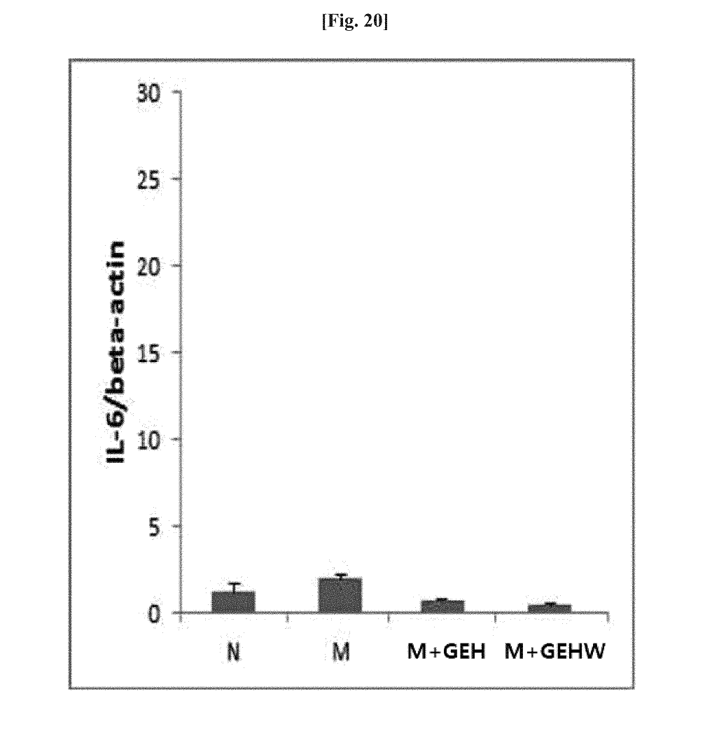

In addition, as illustrated in FIG. 20, a normal group (N) and a metformin-administered group (M) exhibited 1.11 and 1.91 for the gene expression levels of IL-6, respectively. A group co-administered with metformin and a Lonicera japonica extract (30% ethanol) (M+GEH) and a group co-administered with metformin and a Lonicera japonica extract (100% water) (M+GEHW) exhibited decreased gene expression of IL-6, showing values of 0.59 and 0.35, respectively.

### Example 11. Intraperitoneal Insulin Tolerance Test (IPITT) According to Administration of Lonicera japonica Extracts

To identify the effect of co-administration of metformin and a Lonicera japonica extract on diabetes mellitus, 4-week-old OLETF and LETO rats (Otsuka Pharmaceutical, Japan) were purchased and subjected to an 8-week adaptation period, and thereafter the rats were administered with 100 mg/kg of metformin alone or co-administered with 200 mg/kg of a Lonicera japonica extract and 100 mg/kg of metformin. Dietary intakes, body weights, states, and the like were checked weekly, and at 24 weeks, an IPITT was performed using blood collected from the tail veins. After 12 weeks, the rats were sacrificed under anesthesia with an intraperitoneal (IP) injection of Zoletil/Rompun, and fat, each organ sample, and serum were separated. One week prior to the end of the experiments, OLETF/LETO rats were fasted for 15 hours and then administered with 1 U/kg of insulin by intraperitoneal (IP) injection, and then measurement of blood sugar levels was performed using an Accu-Chek blood glucose meter (Roche, USA) on blood samples, which had been collected from the tail vein of each individual by bleeding a small amount of blood at 0, 30, 60, 90, and 120 minutes. The obtained values for blood sugar levels were analyzed using an area under curve (AUC), and the like.

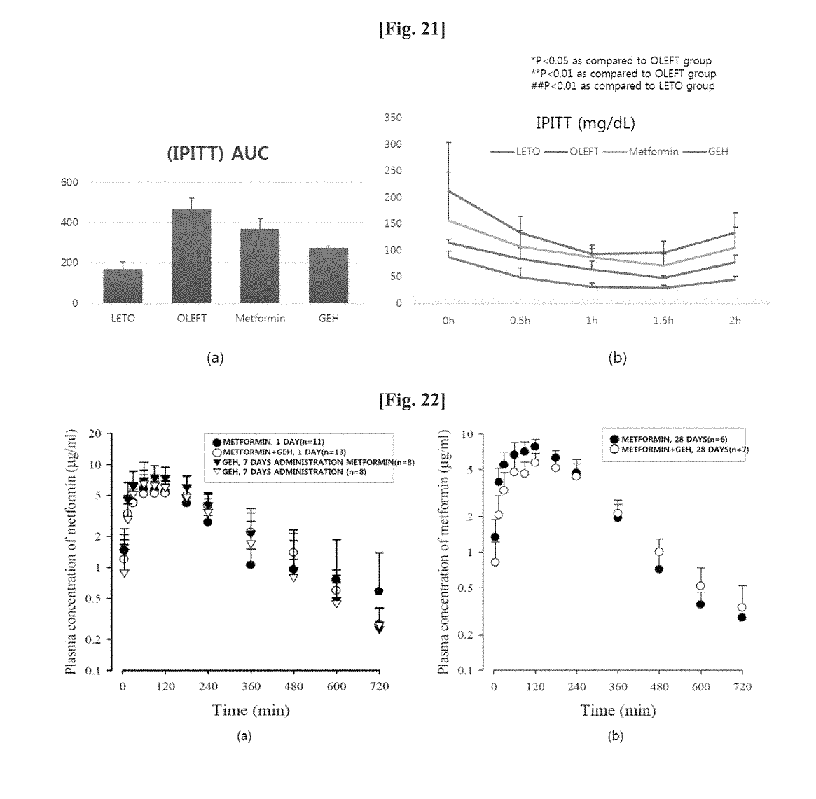

As a result, as illustrated in FIG. 21, an OLEFT group exhibited a higher insulin resistance than a LETO group, and the insulin resistance showed a tendency to decrease through treatment of metformin alone. In addition, when compared to a group administered with metformin alone, groups co-administered with metformin and a Lonicera japonica extract exhibited a significant decrease in insulin resistance.

### Example 12. Measurement of Pharmacokinetic Changes of Metformin by Co-Administration of Metformin and Lonicera japonica Extract

### 12-1. Pharmacokinetic Changes of Metformin According to Period of Co-Administration

A cannula was inserted into an artery of a rat under anesthesia. After awakening from the anesthesia, the rats were orally administered with 100 mg/kg of metformin (a group administered with metformin alone) or with 100 mg/kg of metformin and 200 mg/kg of a Lonicera japonica extract (a group co-administered with metformin and a Lonicera japonica extract). The drugs were administered once, for 7 days, or for 4 weeks according to experimental conditions. After administration, blood was drawn at regular intervals and urine was collected for 24 hours, and at 24 hours, gastrointestinal samples were taken to determine the amount of metformin remaining in the gastrointestinal tract. In addition, a blood concentration profile, urine, and the amount of metformin remaining in the gastrointestinal tract were calculated by quantification using LC/MSMS.

As a result, as illustrated in Table 4 and FIG. 22, when compared to a group administered with metformin alone, no significant changes in pharmacokinetic parameters such as the accumulation effect of metformin were observed in a group co-administered with metformin and a Lonicera japonica extract (once, 7 days, or 4 weeks).

TABLE 4

| | | | Lonicera japonica, | | Lonicera japonica, | |
|---|---|---|---|---|---|---|
| | Single administration | | 7 days administration | | 4 weeks administration | |
| | | Metformin + Lonicera | | Metformin + Lonicera | | Metformin + Lonicera |
| | Metformin | japonica | Metformin | japonica | Metformin | japonica |
| Parameter | (n = 11) | (n = 13) | (n = 8) | (n = 8) | (n = 6) | (n = 7) |
| Body weight | 295 ± 47.7 | 275 ± 49.4 | 291 ± 11.3 | 295 ± 9.30 | 345 ± 32.7 | 356 ± 12.7 |
| (g) | | | | | | |
| AUC_{0-720 min} | 1739 ± 456 | 1940 ± 417 | 1983 ± 546 | 1770 ± 389 | 1984 ± 277 | 1653 ± 318 |
| (μg | | | | | | |
| min/ml) | | | | | | |
| AUC_{0-∞} (μg | 1980 ± 552 | 2001 ± 433 | 2062 ± 508 | 1840 ± 397 | 2140 ± 334 | 1895 ± 275 |
| min/ml) | | | | | | |
| Terminal half- | 171 ± 73.5 | 128 ± 55.1 | 114 ± 40.5 | 139 ± 40.1 | 129 ± 49.1 | 155 ± 34.9 |
| life (min) | | | | | | |
| CL/F (ml/min/ | 54.3 ± 15.4 | 52.5 ± 13.2 | 51.0 ± 11.9 | 56.9 ± 13.9 | 47.6 ± 7.00 | 53.8 ± 8.48 |
| kg) | | | | | | |
| Cₘₐₓ (μg/ml) | 7.65 ± 3.10 | 6.03 ± 1.47 | 8.60 ± 2.30 | 7.54 ± 0.879 | 8.00 ± 1.20 | 6.12 ± 1.21 |
| Tₘₐₓ (min)^{a} | 90 (30-120) | 90 (30-240) | 90 (60-180) | 75 (30-120) | 120 (60-180) | 120 (60-240) |
| CL_{R} (ml/min/kg) | 40.3 ± 13.8 | 43.5 ± 8.71 | 40.5 ± 11.6 | 44.2 ± 12.1 | 37.9 ± 3.60 | 40.1 ± 6.54 |
| Ae_{0-24 h} (% of | 74.0 ± 10.9 | 78.5 ± 8.52 | 79.5 ± 11.4 | 77.6 ± 6.80 | 80.1 ± 5.30 | 75.2 ± 13.3 |
| dose) | | | | | | |
| GI_{24 h} (% of | 5.72 ± 2.05 | 6.36 ± 2.04 | 4.10 ± 2.40 | 6.70 ± 3.30 | 8.80 ± 1.30 | 6.84 ± 4.20 |
| dose) | | | | | | |
| AUC_{0-720 min}/ | 90.2 ± 16.2 | 97.0 ± 1.83 | 95.7 ± 6.10 | 96.1 ± 2.40 | 93.1 ± 6.50 | 88.4 ± 17.3 |
| AUC_{0-∞} | | | | | | |
| (%) | | | | | | |

^{a}Median (ranges)

Whether metformin uptake was changed depending upon combined use of metformin and a Lonicera japonica extract was observed in cell products obtained from OCT transporter expressing cells. Verapamil was used as an inhibitor of OCT1 and 2, while 30 μM and 100 μM verapamil were applied for inhibiting OCT1 and OCT2, respectively, and 10 μM metformin was used as a substrate for OCT1 and 2.

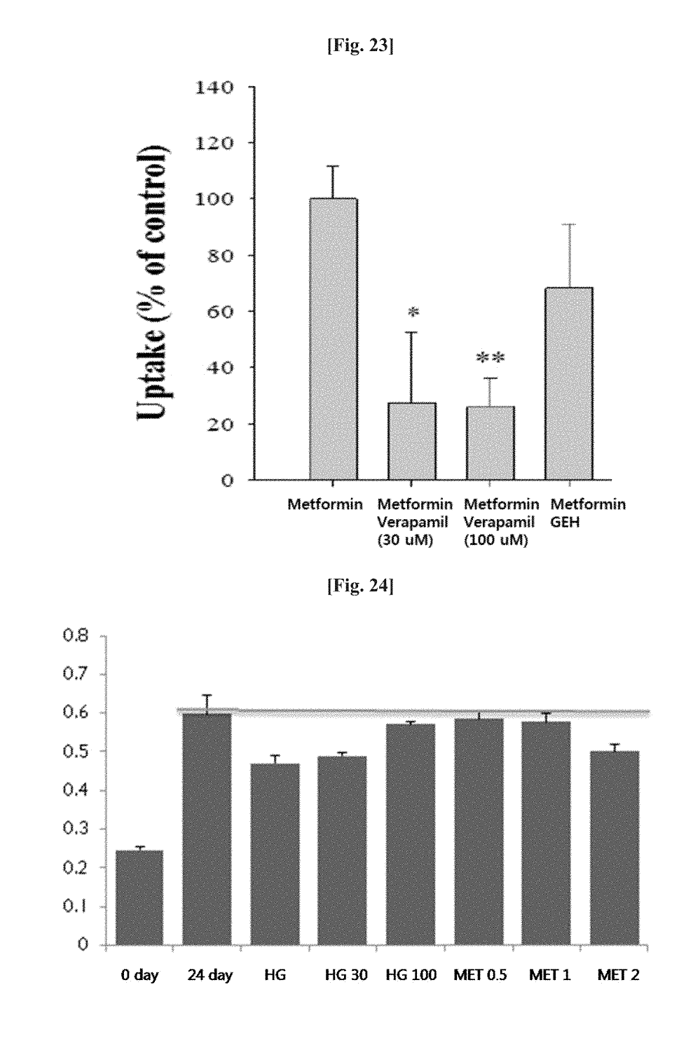

As a result, as illustrated in FIG. 23, when treated with verapamil, an inhibitor of OCT1 and 2, (OCT1: 30 μM verapamil and OCT2: 300 μM verapamil), a significant decrease in metformin uptake was observed in a group administered with metformin alone, whereas no decrease in metformin uptake was observed in a group co-administered with metformin and a Lonicera japonica extract.

Taken together, it was confirmed that co-administration of a Lonicera japonica extract and metformin, an anti-diabetic drug, has no effect on absorption and action of metformin drug itself.

### Example 13. Cytotoxicity Experiments for Scutellaria baicalensis Extracts

Cytotoxicity, using the same method as described in Example 1, was measured according to extraction methods (water extract: HG, 30% ethanol extract: HG30, 100% ethanol extract: HG100), whether metformin was co-administered, and concentration changes of Scutellaria baicalensis extracts (20, 50, 100, 200 μg/ml).

As a result, as illustrated in FIGS. 24 to 26, cytotoxicity was not observed in all groups regardless of extraction method and whether single administration or co-administration with metformin was carried out. In addition, despite an increase in the concentration of Scutellaria baicalensis extracts administered, no cytotoxicity was observed.

### Example 14. Measurement of Changes in Intracellular ROS Activity by Administration of Scutellaria baicalensis Extracts

Using the same method as described in Example 2, changes in intracellular ROS activity by administration of Scutellaria baicalensis extracts were measured.

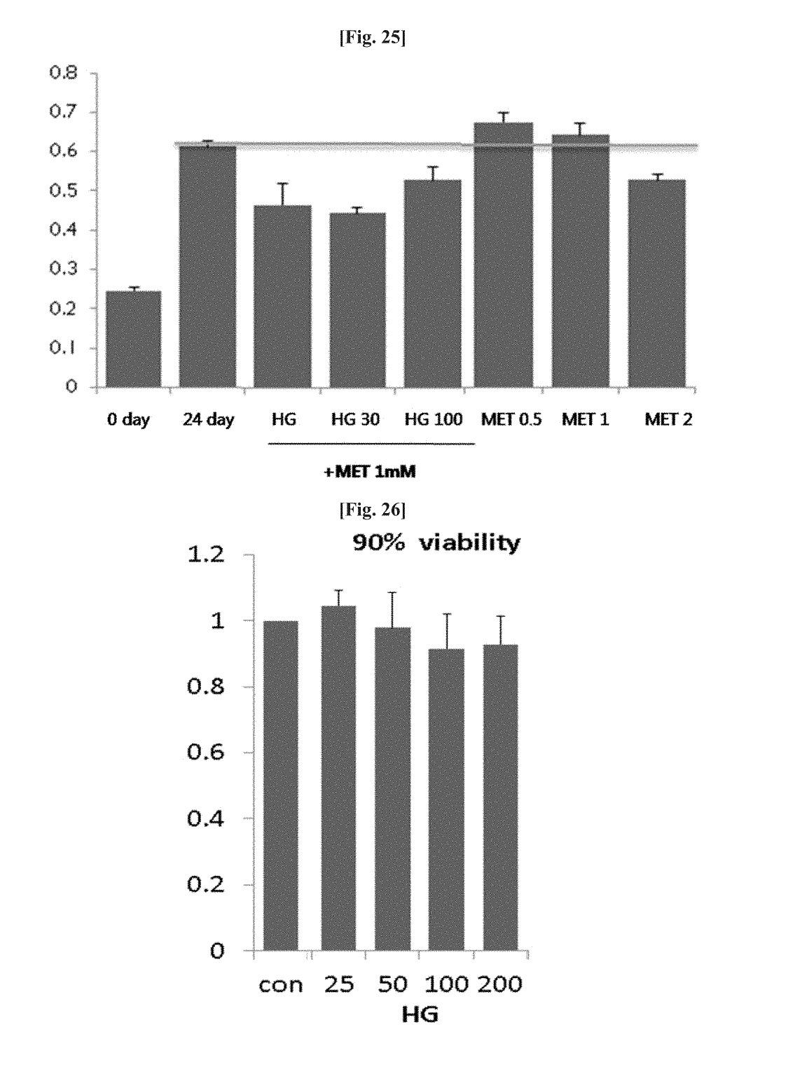

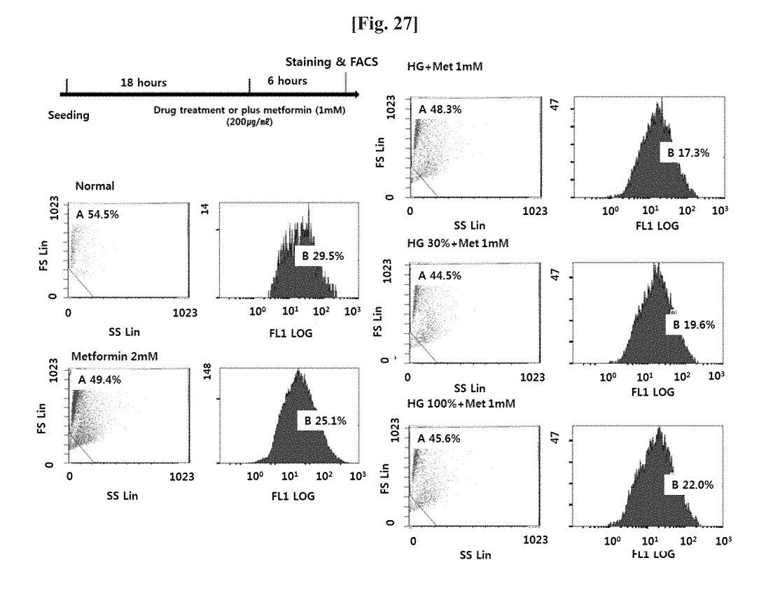

As a result, as illustrated in FIG. 27, a metformin-administered group (Metformin) exhibited a tendency of decreasing intracellular reactive oxygen species (ROS) activity compared to a normal group (Normal). In addition, co-administration of a Scutellaria baicalensis extract and metformin further reduced intracellular ROS activity, and the most significant effect was observed in a Scutellaria baicalensis water extract (HG+Met).

### Example 15. Measurement of DPPH Free Radical Scavenging Activity by Administration of Scutellaria baicalensis Extracts

Using the same method as described in Example 3, depending upon 3 extraction methods (water, 30% ethanol, and 100% ethanol extractions), the DPPH free radical scavenging capacity of a Scutellaria baicalensis extract was measured and IC₅₀ values were calculated.

As a result, BHT, a control group, showed a value of 113.85 μg/ml. In addition, when a Scutellaria baicalensis water extract, a Scutellaria baicalensis 30% ethanol extract, and a Scutellaria baicalensis 100% ethanol extract were administered, as illustrated in the following Table 5, IC₅₀ values were 123.44 μg/ml, 244.36 μg/ml, and 249.47 μg/ml, respectively, demonstrating that these extracts have an excellent free radical scavenging capacity. The most significant effect was observed in a Scutellaria baicalensis water extract (Water extract).

TABLE 5

| IC₅₀ | Scutellaria baicalensis |
|---|---|
| Water extract | 123.44 μg/μl |
| 30% EtoH | 244.36 μg/μl |
| 100% EtoH | 249.47 μg/μl |

### Example 16. Measurement of Capacity of Scutellaria baicalensis Extracts for Inhibiting Nitrogen Monoxide Generation

Using the same method as described in Example 4, the capacity of a Scutellaria baicalensis extract for inhibiting nitrogen monoxide generation was measured.

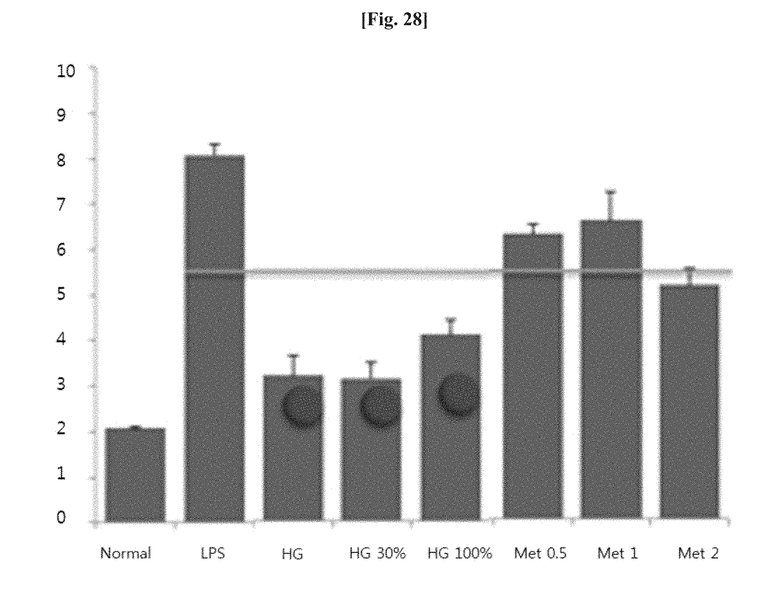

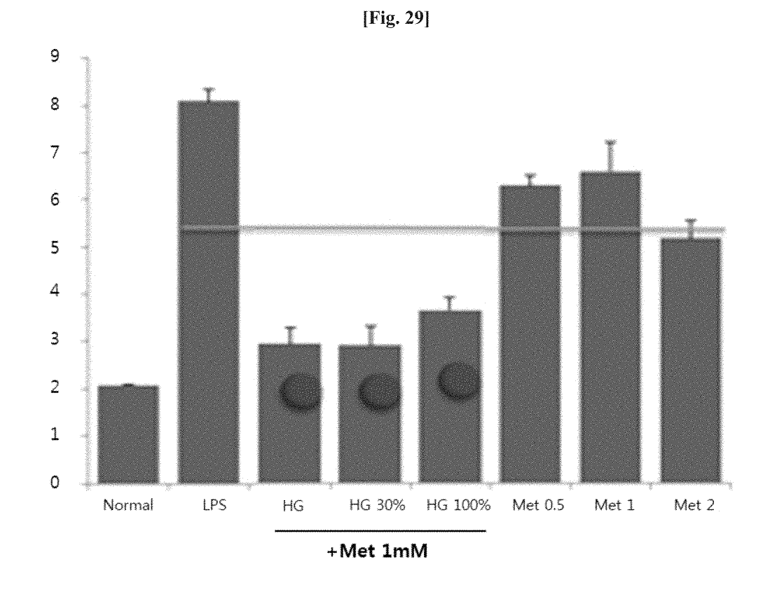

As a result, as illustrated in FIG. 28, the production amount of nitrogen monoxide was decreased in groups administered with metformin alone (Met 0.5, Met 1, and Met 2) compared to an LPS-administered group (LPS). When a Scutellaria baicalensis extract was administered alone, the production amount of nitrogen monoxide was also decreased regardless of extraction method compared to the LPS-administered group (LPS). In addition, as illustrated in FIG. 29, it was confirmed that the production amount of nitrogen monoxide was further decreased in groups co-administered with metformin and a Scutellaria baicalensis extract compared to groups administered with metformin alone.

### Example 17. Confirmation of Inhibitory Effects of Scutellaria baicalensis Extracts on Fat Cell Differentiation

Using the same method as described in Example 5, inhibitory effects of Scutellaria baicalensis extracts on fat cell differentiation were analyzed.

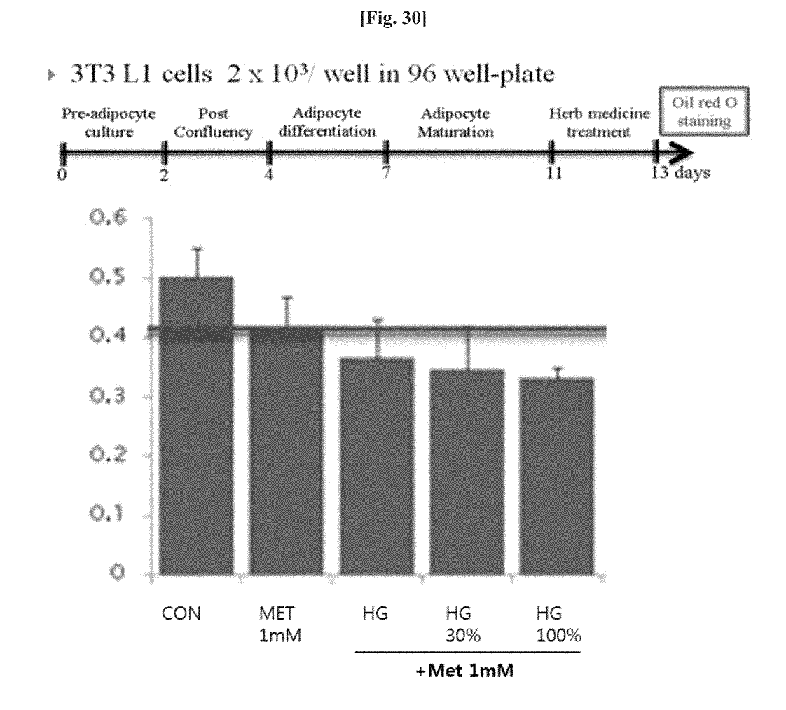

As a result, as illustrated in FIG. 30, a group administered with metformin alone (Met) exhibited a tendency of decreasing lipid formation attributed to differentiation of 3T3-L1 cells, preadipocytes, compared to a control group. In addition, groups co-administered with metformin and a Scutellaria baicalensis extract exhibited an inhibition effect superior to that of the group administered with metformin alone, and a Scutellaria baicalensis 100% ethanol extract (HG 100%+Met) showed the most significant effect.

### Example 18. Glucose Uptake Assay Depending Upon Administration of Scutellaria Baicalensis Extracts

Using the same method as described in Example 6, the capacity of glucose uptake depending upon administration of a Scutellaria baicalensi extract was measured.

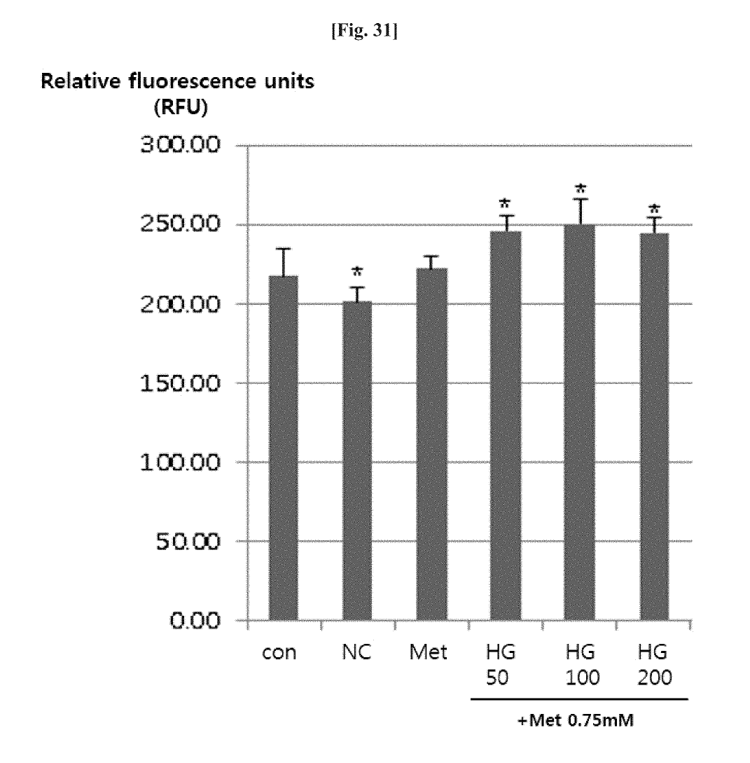

As a result, as illustrated in FIG. 31, a group administered with metformin alone (Met) exhibited an increased capacity of glucose uptake compared to a control group. When comparing the group administered with metformin alone, a group co-administered with a Scutellaria baicalensis extract and metformin exhibited a significant increase in the capacity of glucose uptake.

### Example 19. Confirmation of Changes in Expression Levels of Related Proteins Depending Upon Administration of Scutellaria baicalensis Extracts

3T3-L1 preadipocytes were cultured in DMEM (WELGENE Inc., Korea) containing 10% FBS and 1% penicillin streptomycin (PS) in a CO₂ incubator set to 37° C. with 5% CO₂. The cells were aliquoted to each well of a 6-well plate for cell culture at 8×10⁴ cells/well. To induce cell differentiation, the cells were cultured until reaching 50 to 60% confluence and subsequently, the pre-existing medium were exchanged with a differentiation-inducing DMEM medium containing 0.5 mM IBMX, 1 μM dexamethasone, 10 μg/ml insulin and 10% FBS, and then the cells were cultured for 3 days. After 3 days, the medium of the cell culture was exchanged with a DMEM medium containing 10 μg/ml insulin and 10% FBS and the cell culture was cultured while exchanging the medium every 2 days. At 5 days after differentiation, the cell culture was treated with samples and incubated for 24 hours. The cells in the 6-well plate were washed two times with PBS and subjected to lysis using a RIPA buffer (50 mM Tris-HCl pH 7.4, 1% NP-40, 0.25% sodium deoxycholate, 150 mM NaCl, 1 mM EDTA, 1 mM PMSF, 1 mM NaF, 1 mM sodium, 1 μg/ml aprotinin, leupeptin, pepstatin), and then the lysate was subjected to centrifugation at 12,000 rpm for 20 minutes to obtain a supernatant containing proteins. After performing quantification according to the BCA (Thermo Scientific, USA) method, electrophoresis was carried out on a 10% polyacrylamide gel. After electrophoresis, proteins on the gel were transferred to a PVDF membrane at 200 mA for 90 minutes, and the membrane was treated with a blocking buffer containing 5% skim milk or 5% BSA to reduce background signals due to non-specific proteins and incubated with primary antibodies at 4° C. overnight, and then the membrane was washed three times with TBS-T, in which each washing was performed for 10 minutes. Thereafter, the membrane was treated with secondary antibodies at room temperature for 1 hour and then washed three times with TBS-T, in which each washing was performed for 10 minutes, and the membrane was treated with an ECL (NEURONEX, Korea) solution and subsequently subjected to measurement of protein expression levels using LAS-3000 (FUJIFILM, Japan).

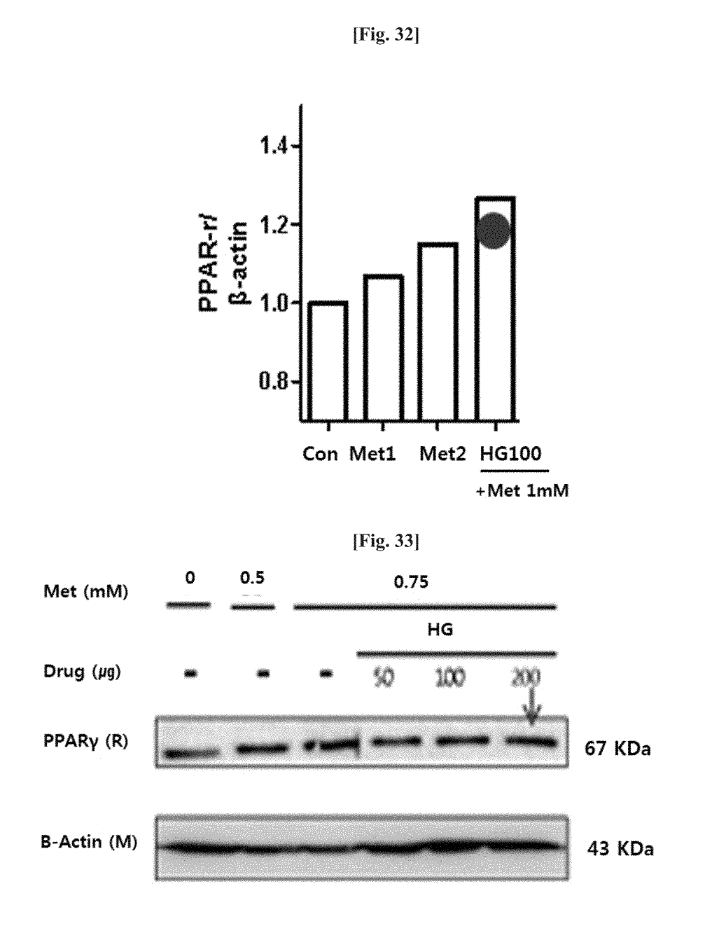

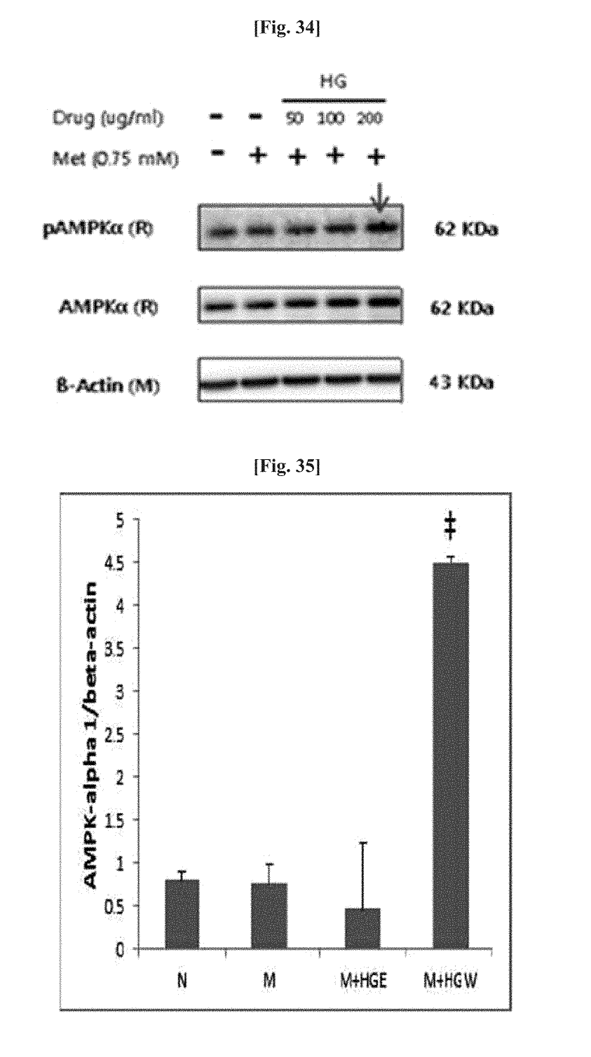

As a result, as illustrated in FIGS. 32 to 34, when comparing groups administered with metformin alone (MET1 and MET2), the expression levels of PPAR-γ and AMPK were significantly increased in a group co-administered with metformin and a Scutellaria baicalensis extract. In Example 19, it has been known that increasing PPAR-γ expression has a positive effect on increasing insulin sensitivity, and that AMPK has a central role in regulating energy metabolism and homeostasis in vivo. Accordingly, the results indicate that co-administration of metformin and a Scutellaria baicalensis extract can further improve an anti-diabetic effect.

### Example 20. Confirmation of Changes in Expression Levels of Related Genes Depending Upon Administration of Scutellaria baicalensis Extracts

20-1. Preparation of Cells and Scutellaria baicalensis Extracts

RAW 264.7 cells, a macrophage cell line, were obtained from the Korean Cell Line Bank (KCLB, Seoul, Korea), and DMEM containing 10% FBS, 2 mM L-glutamine, 100 U/ml penicillin, and 100 μg/ml streptomycin was used as a medium for culturing the RAW 264.7 cells. The cells were cultured in a CO₂ incubator set to 37° C. with 5% CO₂ and 95% O₂. Scutellaria baicalensis extracts (100% water and 30% ethanol extracts) used in the experiments of the present invention were provided from the College of Pharmacy, Dongguk University. Experiments were performed for a total of 4 groups, including a normal group (N), a metformin-administered group (M), a group administered with metformin and a Scutellaria baicalensis extract (30% ethanol extract) (M+HGE), a group administered with metformin and a Scutellaria baicalensis extract (water extract) (M+HGW).

DMEM containing 10% FBS, 2 mM L-glutamine, 100 U/ml penicillin, and 100 μg/ml streptomycin was used as a medium for culturing the RAW 264.7 cells, and the cells were cultured under conditions of 37° C., 5% CO₂, and 90% humidity. The cultured cells were maintained while exchanging the culture medium once every 2 to 3 days. When the cells were fully differentiated, the cell culture was washed with phosphate buffered saline (PBS) and then the cells were detached from a culture dish using a trypsin-EDTA solution. The separated cells were subjected to centrifugation to collect the same, and then the collected cells were mixed with a fresh medium and subcultured.

20-2. Expression of Genes Associated with Anti-Diabetic Effect

To confirm whether combined use of metformin and a Scutellaria baicalensis extract affects the expression of genes associated with an anti-diabetic effect, the gene expression levels of AMPK-α and PPAR-α of RAW 264.7 cells administered with metformin alone (M) and RAW 264.7 cells administered with metformin and a Scutellaria baicalensis extract (30% ethanol or 100% water) were compared using real-time PCR.

Total RNA was separated and purified using TRIsure (Bioline, USA) according to a protocol. 1 μg of total RNAs was subjected to a reverse transcription reaction using a cDNA synthesis kit (Sprint™RT Complete Oligo-(dT)18, Clontech, Mountain View, Calif., USA) according to a protocol for synthesizing first strand cDNA. The produced RT-PCR sample was subjected to real-time PCR, in which the final reaction volume was adjusted to 20 μl and Light Cycler-Fast Start DNA Master SYBR Green (Roche Applied Science, Indianapolis, Ind., USA) and a Light Cycler instrument (Roche Applied Science) were used.

DNA sequences of primers used in Example 20-2 are as follows.

TABLE 6

| Genus | | | |
|---|---|---|---|
| Specific | | | Annealing |
| Primers | Direction | Sequence | temp. |
| Beta-actin | F | 5′-GCAAGTGCTTCTAGGCGGAC-3′ | 52° C. |
| | | (SEQ ID NO. 1) | |
| | R | 5′-AAGAAAGGGTGTAAAACGCAGC-3′ | |
| | | (SEQ ID NO. 2) | |
| AMPK alpha 1 | F | 5′-AAGCCGACCCAATGACATCA-3′ | 49° C. |
| | | (SEQ ID NO. 3) | |
| | R | 5′-CTTCCTTCGTACACGCAAAT-3′ | |
| | | (SEQ ID NO. 4) | |
| PPAR-alpha | F | 5′-GCCTGTCTGTCGGGATGT-3′ | 50° C. |
| | | (SEQ ID NO. 5) | |
| | R | 5′-GGCTTCGTGGATTCTCTTG-3′ | |
| | | (SEQ ID NO. 6) | |

PCR amplification was performed according to PCR steps, consisting of a pre-incubation step at 95° C. for 10 minutes and 35 (for beta-actin) or 45 (for C/EBPa) cycles of amplification (denaturation at 95° C. for 10 seconds, annealing at 52° C. for 10 seconds, and extension at 72° C. for 15 seconds). Total RNA was separated and purified using TRIsure (Bioline, USA) according to a protocol. 1 μg of total RNAs was subjected to a reverse transcription reaction using a cDNA synthesis kit (Sprint™RT Complete Oligo-(dT)18, Clontech, Mountain View, Calif., USA) according to a protocol for synthesizing first strand cDNA. The produced RT-PCR sample was subjected to real-time PCR, in which the final reaction volume was adjusted to 20 μl and Light Cycler-Fast Start DNA Master SYBR Green (Roche Applied Science, Indianapolis, Ind., USA) and a Light Cycler instrument (Roche Applied Science) were used.

As a result, as illustrated in FIG. 35, a normal group (N) and a metformin-administered group (M) showed 0.80 and 0.76 for the gene expression levels of AMPK-α, respectively. A group co-administered with metformin and a Scutellaria baicalensis extract (100% water) (M+HGW) exhibited a significant increase in AMPK-α gene expression, showing a value of 4.50.

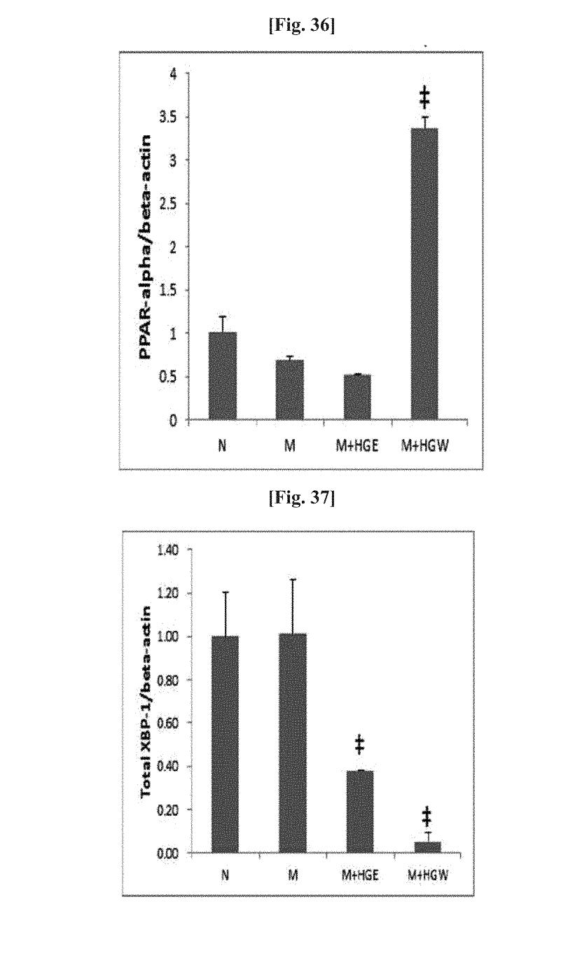

In addition, as illustrated in FIG. 36, a normal group (N) and a metformin-administered group (M) exhibited 1.01 and 0.68 for the gene expression levels of PPAR-α, respectively. A group co-administered with metformin and a Scutellaria baicalensis extract (100% water) (M+HGW) exhibited increased gene expression of PPAR-α, showing a value of 3.38.

20-3. Expression of Genes Associated with Side Effects of Metformin

To identify the effect of co-administration of metformin and a Scutellaria baicalensis extract on expression of genes, which are associated with side effects caused by metformin, the gene expression levels of XBP-1, TNF-α, and IL-6 of RAW 264.7 cells administered with metformin alone (M) and RAW 264.7 cells administered with metformin and a Scutellaria baicalensis extract (30% ethanol or 100% water) were compared using real-time PCR.

To identify the gene expression levels of XBP-1, TNF-α, and IL-6, real-time PCR was performed according to the same method as described in Example 3 except primers.

DNA sequences of primers used in Example 20-3 are as follows.

TABLE 7

| Genus | | | |
|---|---|---|---|
| Specific | | | Annealing |
| Primers | Direction | Sequence | temp. |
| beta-actin | F | 5′-GCAAGTGCTTCTAGGCGGAC-3′ | 52° C. |
| | | (SEQ ID NO. 1) | |
| | R | 5′-AAGAAAGGGTGTAAAACGCAGC-3′ | |
| | | (SEQ ID NO. 2) | |
| XBP-1 | F | 5′-TGGCCGGGTCTGCTGAGTCCG-3′ | 51° C. |
| | | (SEQ ID NO. 9) | |
| | R | 5′-GTCCATGGGAAGATGTTCTGG-3′ | |
| | | (SEQ ID NO. 10) | |
| TNF-alpha | F | 5′-GAACTGGCAGAAGAGGCACT-3′ | 52° C. |
| | | (SEQ ID NO. 11) | |
| | R | 5′-AGGGTCTGGGCCATAGAACT-3′ | |
| | | (SEQ ID NO. 12) | |
| IL-6 | F | 5′-AGTTGCCTTCTTGGGACTGA-3′ | 49° C. |
| | | (SEQ ID NO. 13) | |
| | R | 5′-CAGAATTGCCATTGCACAAC-3′ | |
| | | (SEQ ID NO. 14) | |

As a result, as illustrated in FIG. 37, a normal group (N) and a metformin-administered group (M) exhibited 1.00 and 1.01 for the gene expression levels of XBP-1, respectively. A group co-administered with metformin and a Scutellaria baicalensis extract (30% ethanol) (M+HGE) and a group co-administered with metformin and a Scutellaria baicalensis extract (100% water) (M+HGW) exhibited decreased gene expression of XBP-1, showing values of 0.38 and 0.05, respectively.

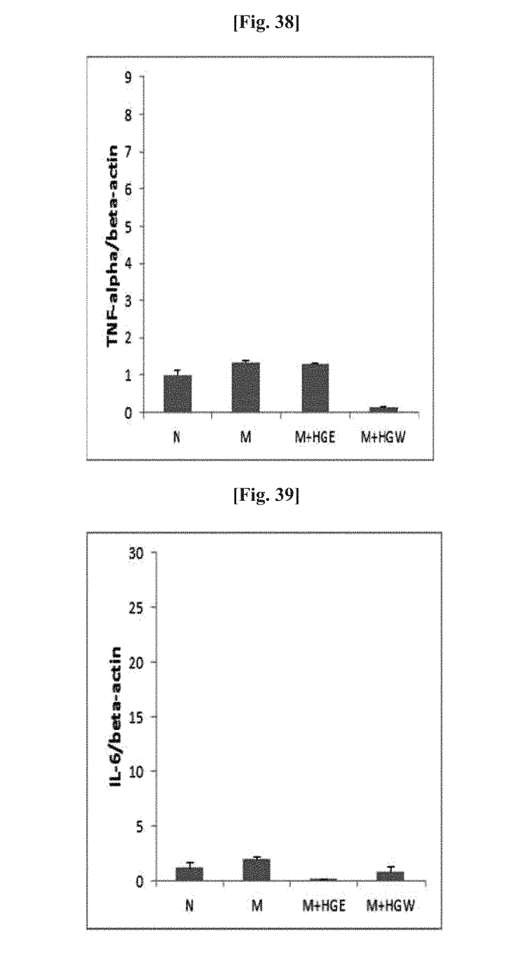

In addition, as illustrated in FIG. 38, a normal group (N) and a metformin-administered group (M) exhibited 1.01 and 1.34 for the gene expression levels of TNF-α, respectively. A group co-administered with metformin and a Scutellaria baicalensis extract (100% water) (M+HGW) exhibited decreased gene expression of TNF-α.

In addition, as illustrated in FIG. 39, a normal group (N) and a metformin-administered group (M) exhibited 1.11 and 1.91 for the gene expression levels of IL-6, respectively. A group co-administered with metformin and a Scutellaria baicalensis extract (30% ethanol) (M+HGE) and a group co-administered with metformin and a Scutellaria baicalensis extract (100% water) (M+HGW) exhibited decreased gene expression of IL-6, showing values of 0.12 and 0.74, respectively.

### Example 21. Intraperitoneal Insulin Tolerance Test (IPITT) According to Administration of Scutellaria baicalensis Extracts

Using the same method as described in Example 11, an IPITT was performed to identify the effect of administration of Scutellaria baicalensis extracts on insulin tolerance.

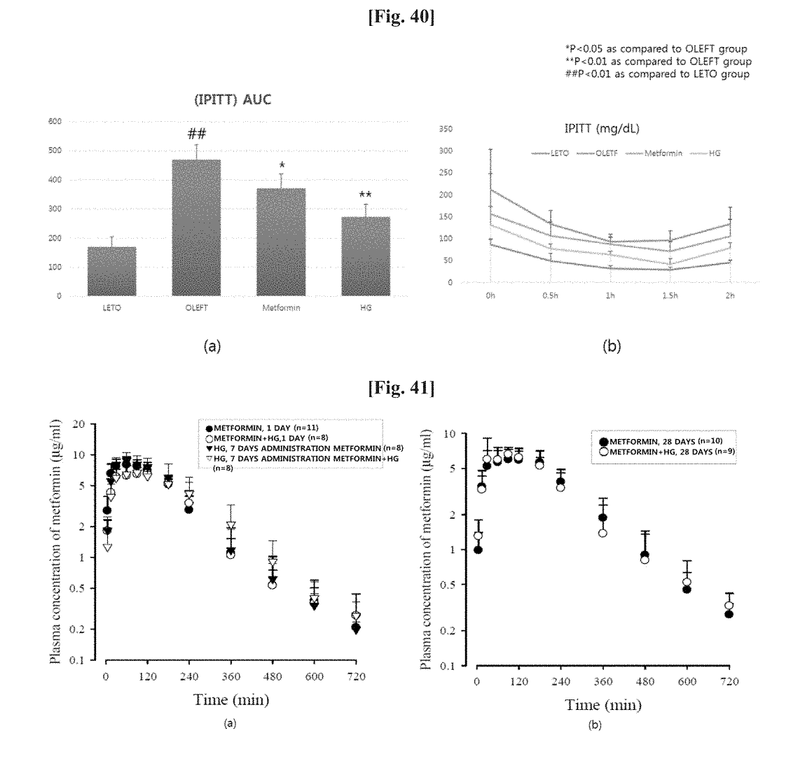

As a result, as illustrated in FIG. 40, an OLEFT group exhibited a higher insulin resistance than a LETO group, and the insulin resistance showed a tendency to decrease through treatment of metformin alone. In addition, when compared to a group administered with metformin alone, groups co-administered with metformin and a Scutellaria baicalensis extract exhibited a significant decrease in insulin resistance.

### Example 22. Measurement of Pharmacokinetic Changes of Metformin by Co-Administration of Metformin and Scutellaria baicalensis Extract

### 22-1. Pharmacokinetic Changes of Metformin According to Period of Co-Administration

Using the same method as described in Example 12-1, pharmacokinetic changes of metformin according to the period of co-administration were measured.

As a result, as illustrated in Table 8 and FIG. 41, although a tendency of slightly decreasing Cₘₐₓ was observed in a group co-administered with metformin and a Scutellaria baicalensis extract (once, 7 days, or 4 weeks) compared to a group administered with metformin alone, no significant changes in AUC were observed in the co-administrated group. Thus, no significant changes in pharmacokinetic parameters such as the accumulation effect of metformin were observed in a group co-administered with metformin and a Scutellaria baicalensis extract (once, 7 days, or 4 weeks).

TABLE 8

| | | | Scutellaria baicalensis, | | Scutellaria baicalensis, | |
|---|---|---|---|---|---|---|
| | Single administration | | 7 days administration | | 4 weeks administration | |
| | | Metformin + | | Metformin + | | Metformin + |
| | | Scutellaria | | Scutellaria | | Scutellaria |
| | Metformin | baicalensis | Metformin | baicalensis | Metformin | baicalensis |
| Parameter | (n = 11) | (n = 8) | (n = 8) | (n = 8) | (n = 10) | (n = 9) |
| Body | 253 ± 21.6 | 264 ± 31.1 | 274 ± 6.94 | 277 ± 15.8 | 351 ± 27.5 | 286 ± 169 |
| weight (g) | | | | | | |
| AUC_{0-720 min} | 1834 ± 198 | 1695 ± 315 | 2065 ± 460 | 1922 ± 311 | 1865 ± 312 | 1745 ± 519 |
| (μg | | | | | | |
| min/ml) | | | | | | |
| AUC_{0-∞} (μg | 1940 ± 209 | 1790 ± 316 | 2131 ± 457 | 1985 ± 313 | 1940 ± 296 | 1875 ± 492 |
| min/ml) | | | | | | |
| Terminal | 256 ± 141 | 210 ± 75.7 | 165 ± 71.4 | 152 ± 59.1 | 187 ± 102 | 288 ± 180 |
| half- | | | | | | |
| life (min) | | | | | | |
| CL/F (ml/min/ | 52.1 ± 6.01 | 57.4 ± 9.81 | 48.7 ± 9.86 | 51.4 ± 7.65 | 52.6 ± 8.17 | 55.8 ± 12.5 |
| kg) | | | | | | |
| Cₘₐₓ (μg/ | 8.60 ± 1.13 | 6.93 ± 0.926^{a} | 9.21 ± 1.44 | 7.54 ± 0.879^{a} | 7.14 ± 1.05 | 6.76 ± 0.189 |
| ml) | | | | | | |
| Tₘₐₓ (min)^{a} | 60 (30-120) | 60 (30-120) | 60 (30-120) | 90 (30-180) | 90 (30-180) | 90 (60-180) |
| CL_{R} (ml/min/ | 35.03 ± 6.33 | 35.9 ± 4.02 | 34.2 ± 9.80 | 38.1 ± 4.31 | 38.6 ± 9.09 | 117 ± 143 |
| kg) | | | | | | |
| Ae_{0-24 h} (% | 80.5 ± 12.1 | 71.02 ± 11.1 | 77.3 ± 18.6 | 83.03 ± 8.11 | 81.8 ± 15.4 | 75.8 ± 17.5 |
| of dose) | | | | | | |
| GI_{24 h} (% of | 12.9 ± 2.70 | 10.8 ± 4.49 | 6.52 ± 3.31 | 5.09 ± 1.64 | 9.21 ± 4.88 | 10.7 ± 3.31 |
| dose) | | | | | | |
| AUC_{0-720 min}/ | 94.6 ± 3.36 | 94.7 ± 4.34 | 96.8 ± 2.35 | 96.8 ± 1.27 | 95.9 ± 2.08 | 92.6 ± 4.12 |
| AUC_{0-∞} | | | | | | |
| (%) | | | | | | |

^{a}P < 0.05 compared with metformin
^{b}Median (ranges)

### 22-2. Changes in Metformin Uptake by Inhibition of OCT 1 and OCT 2

Using the same method as described in Example 12-2, changes in metformin uptake by inhibition of OCT 1 and 2 were measured.

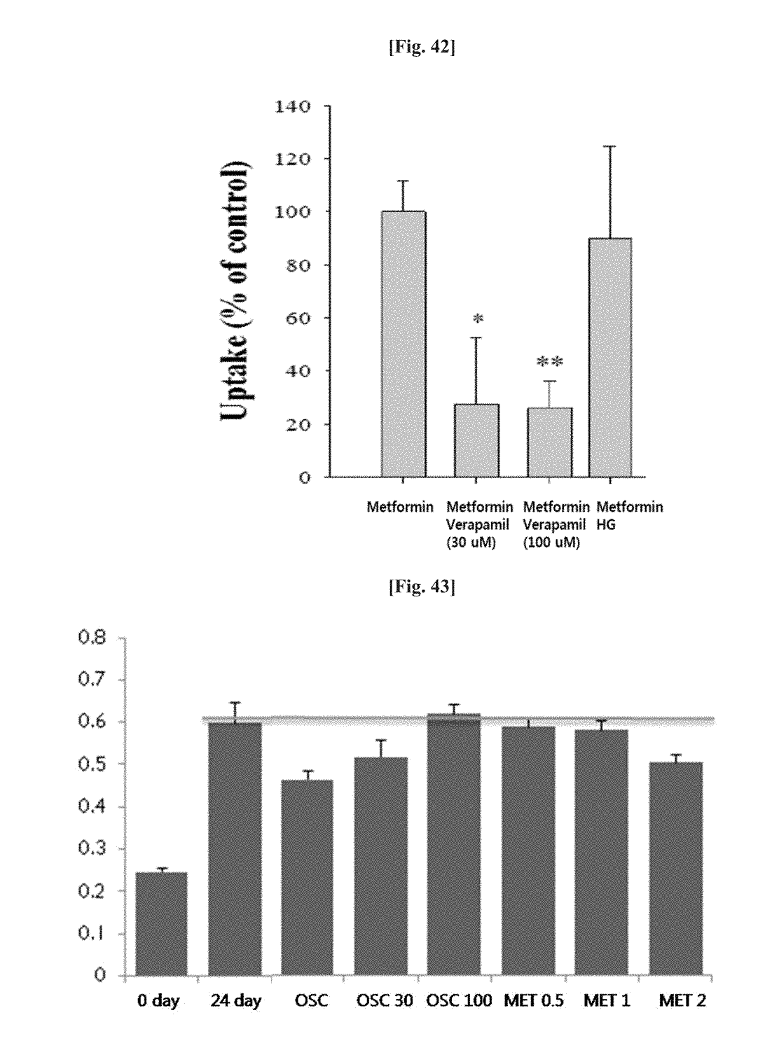

As a result, as illustrated in FIG. 42, when treated with verapamil, an inhibitor of OCT1 and 2, (OCT1: 30 μM verapamil and OCT2: 500 μM verapamil), a significant decrease in metformin uptake was observed in a group administered with metformin alone, whereas no decrease in metformin uptake was observed in a group co-administered with metformin and a Scutellaria baicalensis extract.

Taken together, it was confirmed that co-administration of a Scutellaria baicalensis extract and metformin, an anti-diabetic drug, has no effect on absorption and action of metformin drug itself.

### Example 23. Cytotoxicity Experiments for Houttuynia cordata Extracts

Cytotoxicity, using the same method as described in Example 1, was measured according to extraction methods (water extract: OSC, 30% ethanol extract: OSC30, 100% ethanol extract: OSC100), whether metformin was co-administered, and concentration changes of Houttuynia cordata extracts (20, 50, 100, 200 μg/ml).

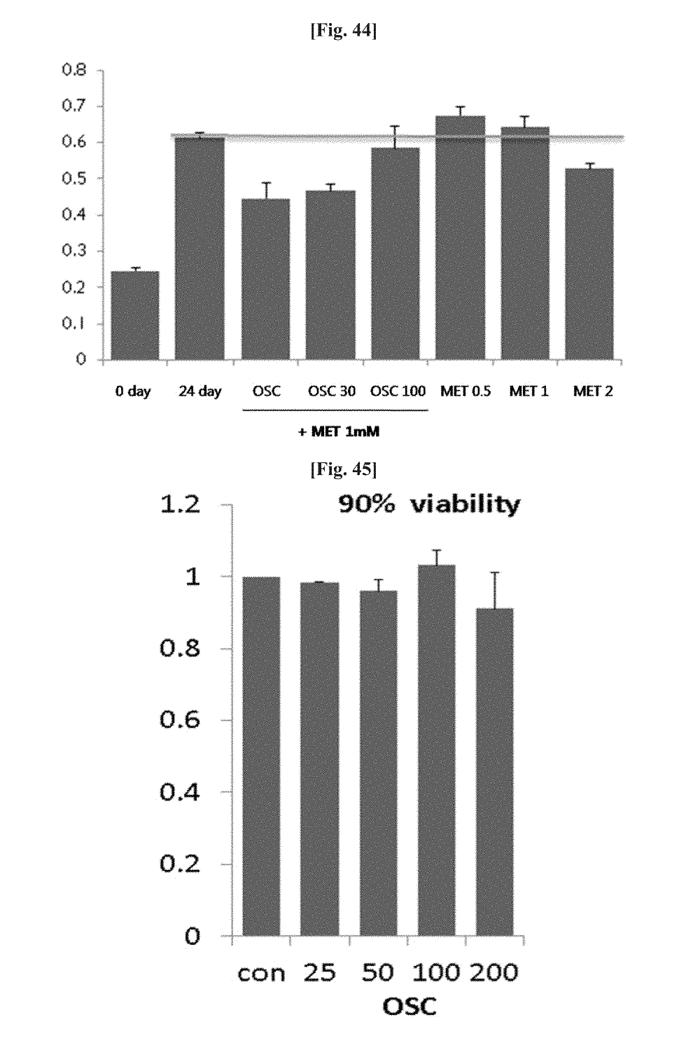

As a result, as illustrated in FIGS. 43 to 45, cytotoxicity was not observed in all groups regardless of extraction method and whether single administration or co-administration with metformin was carried out. In addition, despite an increase in the concentration of Houttuynia cordata extracts administered, no cytotoxicity was observed.

### Example 24. Measurement of Changes in Intracellular ROS Activity by Administration of Houttuvnia cordata Extracts

Using the same method as described in Example 2, changes in intracellular ROS activity by administration of Houttuynia cordata extracts were measured.

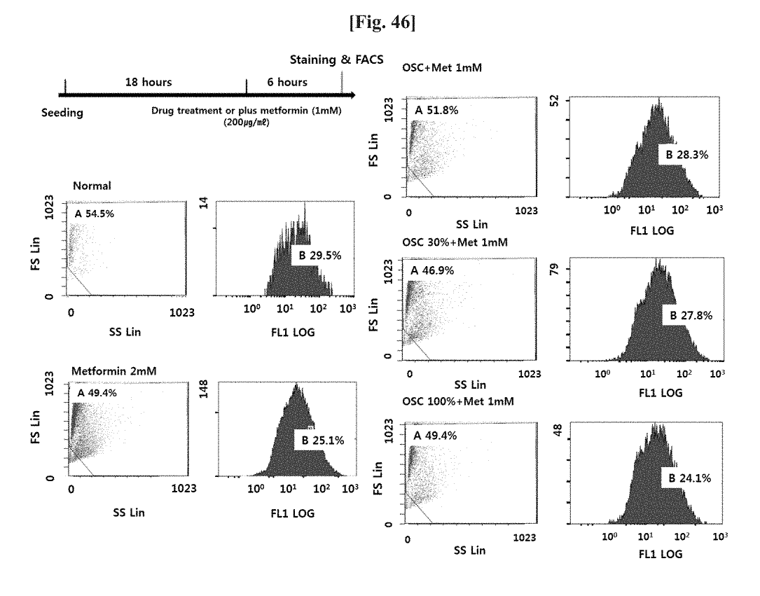

As a result, as illustrated in FIG. 46, a metformin-administered group (Metformin) exhibited a tendency of decreasing intracellular reactive oxygen species (ROS) activity compared to a normal group (Normal). In addition, co-administration of a Houttuynia cordata extract and metformin further reduced intracellular ROS activity, and the most significant effect was observed in a Houttuynia cordata 100% ethanol extract (OSC 100%+Met).

### Example 25. Measurement of DPPH Free Radical Scavenging Activity by Administration of Houttuynia cordata Extracts

Using the same method as described in Example 3, depending upon 3 extraction methods (water, 30% ethanol, and 100% ethanol extractions), the DPPH free radical scavenging capacity of a Houttuynia cordata extract was measured and IC₅₀ values were calculated.

As a result, BHT, a control group, showed a value of 113.85 μg/ml. In addition, when a Houttuynia cordata water extract, a Houttuynia cordata 30% ethanol extract, and a Houttuynia cordata 100% ethanol extract were administered, as illustrated in the following Table 9, IC₅₀ values were 239.80 μg/ml, 246.10 μg/ml, and 293.11 μg/ml, respectively, demonstrating that these extracts have an excellent free radical scavenging capacity. The most significant effect was observed in a Houttuynia cordata water extract (Water extract).

TABLE 9

| IC₅₀ | Houttuynia cordata |
|---|---|
| Water extract | 239.80 μg/μl |
| 30% EtoH | 246.10 μg/μl |
| 100% EtoH | 293.11 μg/μl |

### Example 26. Measurement of Capacity of Houttuynia cordata Extracts for Inhibiting Nitrogen Monoxide Generation

Using the same method as described in Example 4, the capacity of a Houttuynia cordata extract for inhibiting nitrogen monoxide generation was measured.

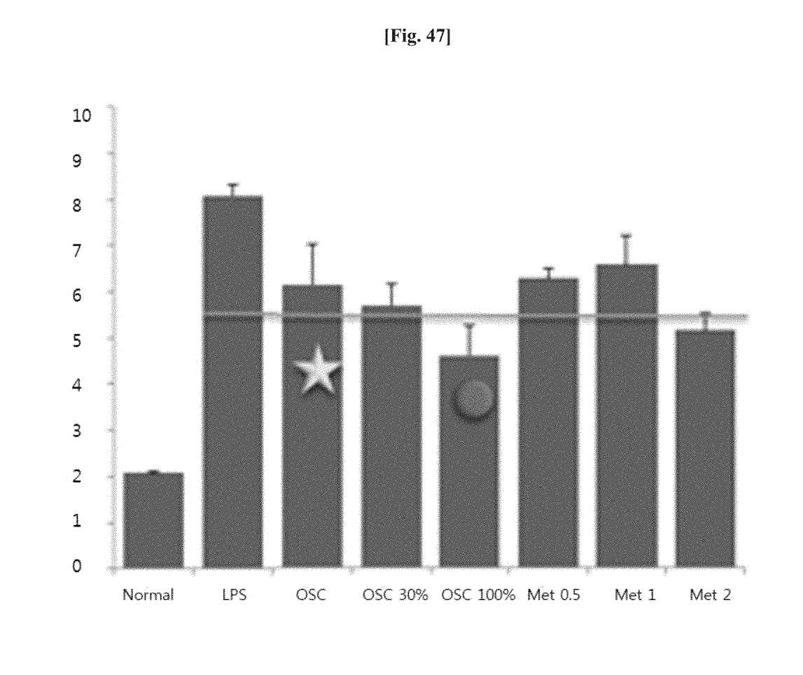

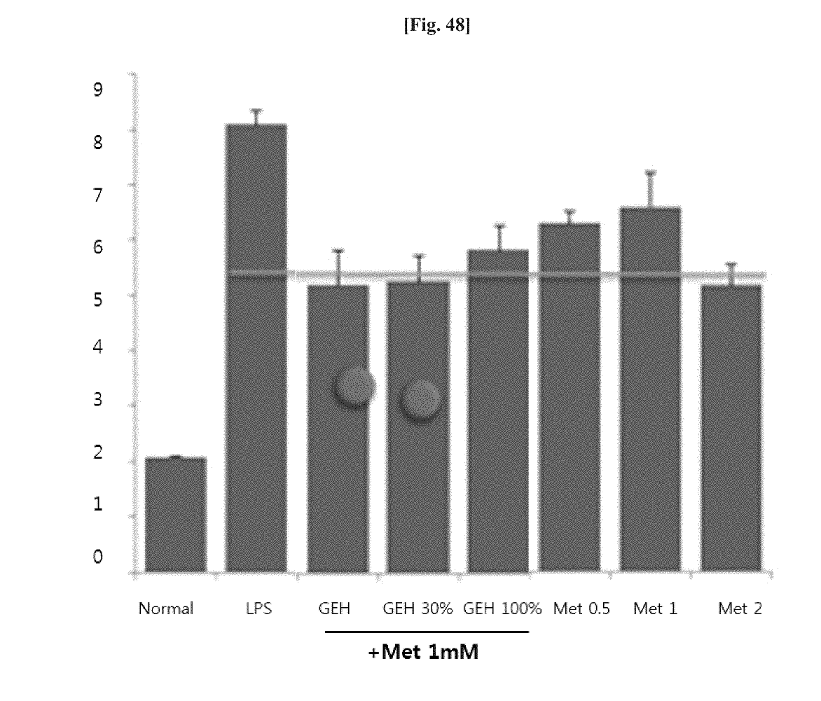

As a result, as illustrated in FIG. 47, the production amount of nitrogen monoxide was decreased in groups administered with metformin alone (Met 0.5, Met 1, and Met 2) compared to an LPS-administered group (LPS). When a Houttuynia cordata extract was administered alone, the production amount of nitrogen monoxide was also decreased regardless of extraction method compared to an LPS-administered group (LPS). In addition, as illustrated in FIG. 48, the production amount of nitrogen monoxide was further decreased in groups co-administered with metformin and a Houttuynia cordata extract compared to groups administered with metformin alone, indicating that a synergistic effect can be obtained when using a combination of metformin and a Houttuynia cordata extract.

### Example 27. Confirmation of Inhibitory Effects of Houttuynia cordata Extracts on Fat Cell Differentiation

Using the same method as described in Example 5, inhibitory effects of Houttuynia cordata extracts on fat cell differentiation were analyzed.

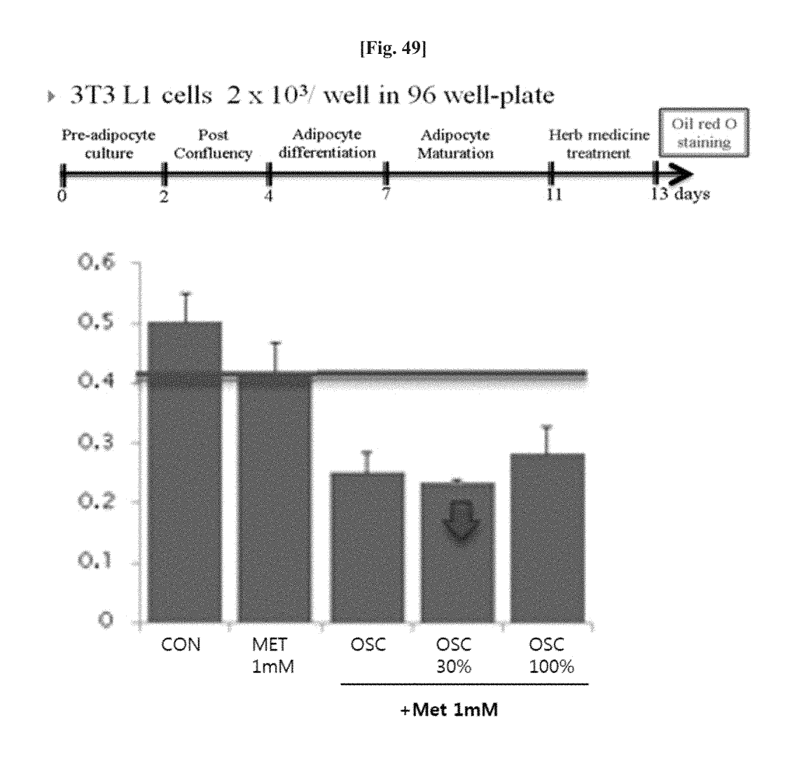

As a result, as illustrated in FIG. 49, a group administered with metformin alone (Met) exhibited a tendency of decreasing lipid formation attributed to differentiation of 3T3-L1 cells, preadipocytes, compared to a control group. In addition, groups co-administered with metformin and a Houttuynia cordata extract exhibited an inhibition effect superior to that of the group administered with metformin alone, and a Houttuynia cordata 30% ethanol extract (OSC 30%+Met) showed the most significant effect.

### Example 28. Glucose Uptake Assay According to Administration of Houttuynia cordata Extracts

Using the same method as described in Example 6, the capacity of glucose uptake depending upon administration of a Houttuynia cordata extract was measured.

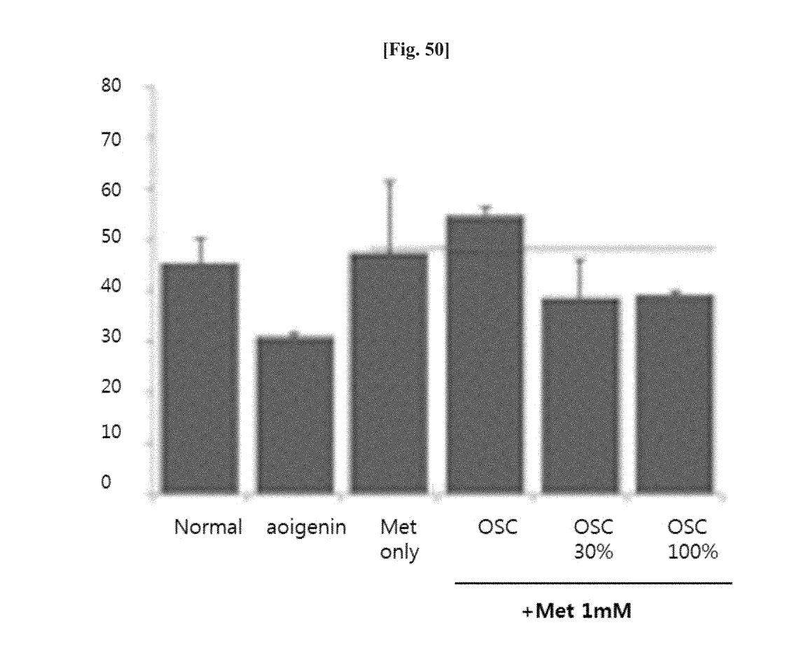

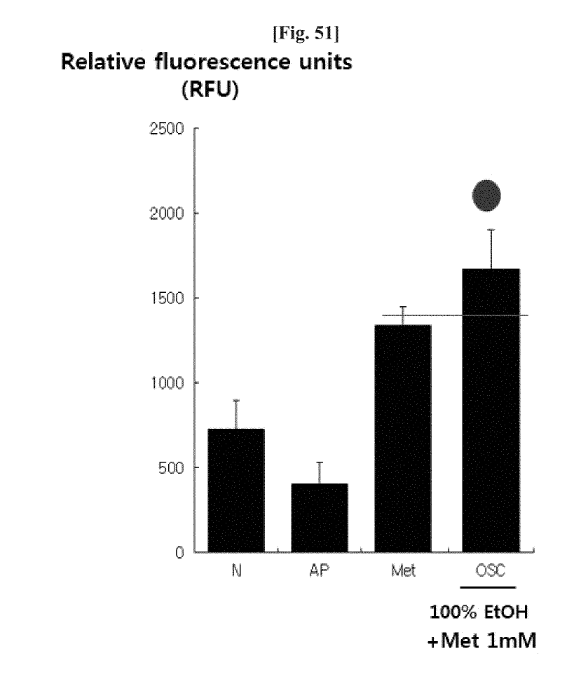

As a result, as illustrated in FIGS. 50 and 51, groups administered with metformin alone (Met) exhibited an increased capacity of glucose uptake compared to a control group. When comparing the group administered with metformin alone, a group co-administered with a Houttuynia cordata water extract and metformin (OSC) and a group co-administered with a Houttuynia cordata 100% ethanol extract and metformin (OSC 100) exhibited significant increases in the capacity of glucose uptake. In addition, as illustrated in FIG. 52, it was confirmed that as the concentration of a Houttuynia cordata extract co-administrated with metformin was increased (50, 100, 200 μg/ml), the capacity of glucose uptake was increased.

### Example 29. Insulin Secretion Assay Depending Upon Administration of Houttuynia Cordata Extracts

Using the same method as described in Example 7, the capacity of insulin secretion depending upon administration of Houttuynia cordata extracts was measured.

As a result, as illustrated in FIG. 53, while significant changes in the amounts of insulin secretion were not observed in groups administered with metformin alone (MET1 and MET2), significant changes in the amounts of insulin secretion were observed in groups co-administered with metformin and each of the Houttuynia cordata extracts. The group co-administrated with metformin and a Houttuynia cordata 30% ethanol extract exhibited the most significant change.

### Example 30. Insulin Resistance Assay Depending Upon Administration of Houttuvnia Cordata Extracts

Using the same method as described in Example 8, insulin resistance depending upon administration of Houttuynia cordata extracts was measured.

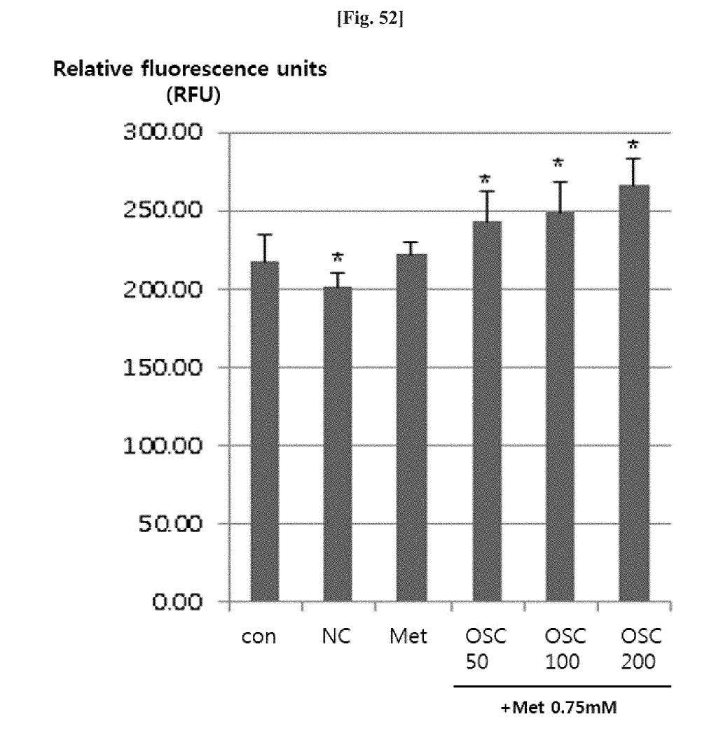

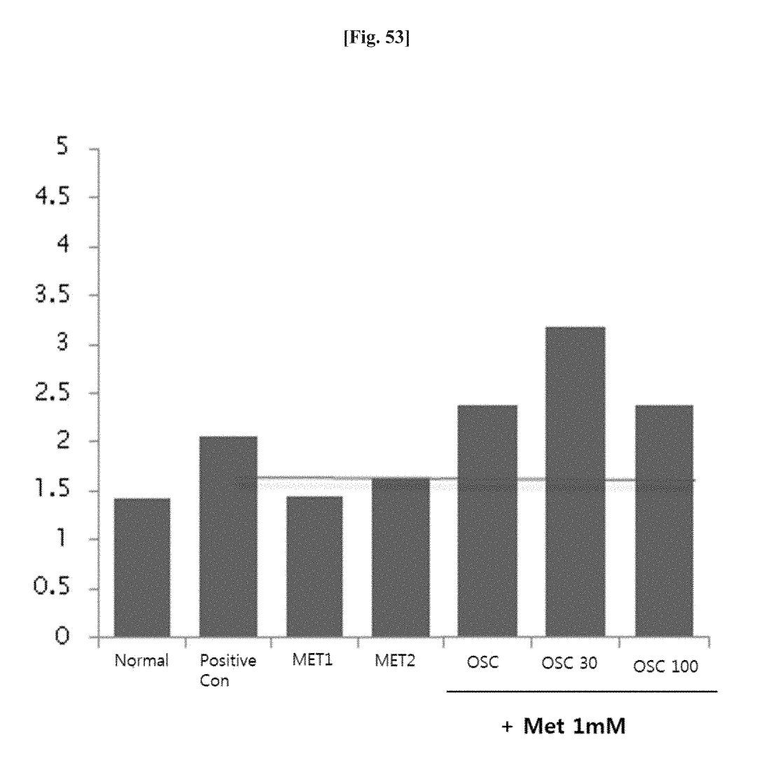

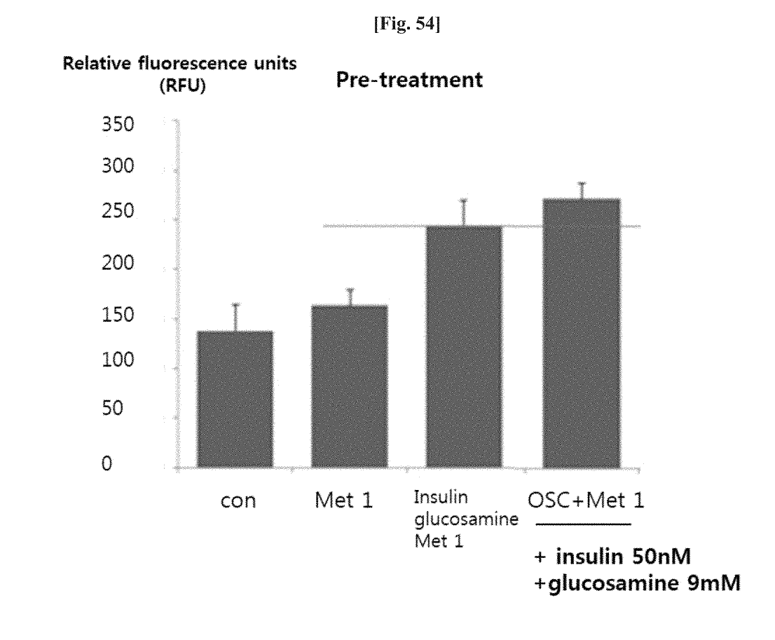

As a result, as illustrated in FIG. 54, under a condition of treating with insulin and glucosamine, a group co-administered with a Houttuynia cordata extract and metformin exhibited superior glucose uptake compared to a group administered with metformin alone, indicating that a synergistic effect on improving insulin resistance can be obtained when using a combination of metformin and a Houttuynia cordata extract.

### Example 31. Confirmation of Changes in Expression Levels of Related Proteins Depending Upon Administration of Houttuvnia cordata Extracts

3T3-L1 preadipocytes were cultured in DMEM (WELGENE Inc., Korea) containing 10% FBS and 1% penicillin streptomycin (PS) in a CO₂ incubator set to 37° C. with 5% CO₂. The cells were aliquoted to each well of a 6-well plate for cell culture at 8×10⁴ cells/well. To induce cell differentiation, the cells were cultured until reaching 50 to 60% confluence and subsequently, the pre-existing medium was exchanged with a differentiation-inducing DMEM medium containing 0.5 mM IBMX, 1 μM dexamethasone, 10 μg/ml insulin and 10% FBS, and then the cells were cultured for 3 days. After 3 days, the medium of the cell culture was exchanged with a DMEM medium containing 10 μg/ml insulin and 10% FBS and the cell culture was cultured while exchanging the medium every 2 days. At 5 days after differentiation, the cell culture was treated with samples and incubated for 24 hours. The cells in the 6-well plate were washed two times with PBS and subjected to lysis using a RIPA buffer (50 mM Tris-HCl pH 7.4, 1% NP-40, 0.25% sodium deoxycholate, 150 mM NaCl, 1 mM EDTA, 1 mM PMSF, 1 mM NaF, 1 mM sodium, 1 μg/ml aprotinin, leupeptin, pepstatin), and then the lysate was subjected to centrifugation at 12,000 rpm for 20 minutes to obtain a supernatant containing proteins. After performing quantification according to the BCA (Thermo Scientific, USA) method, electrophoresis was carried out on a 10% polyacrylamide gel. After electrophoresis, proteins on the gel were transferred to a PVDF membrane at 200 mA for 90 minutes, and the membrane was treated with a blocking buffer containing 5% skim milk or 5% BSA to reduce background signals due to non-specific proteins and incubated with primary antibodies at 4° C. overnight, and then the membrane was washed three times with TBS-T, in which each washing was performed for 10 minutes. Thereafter, the membrane was treated with secondary antibodies at room temperature for 1 hour and then washed three times with TBS-T, in which each washing was performed for 10 minutes, and the membrane was treated with an ECL (NEURONEX, Korea) solution and subsequently subjected to measurement of protein expression levels using LAS-3000 (FUJIFILM, Japan).

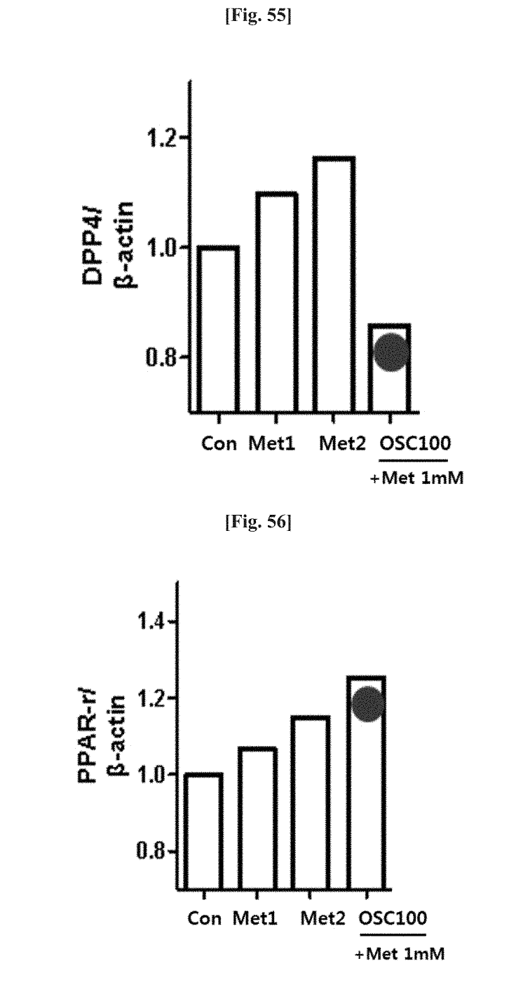

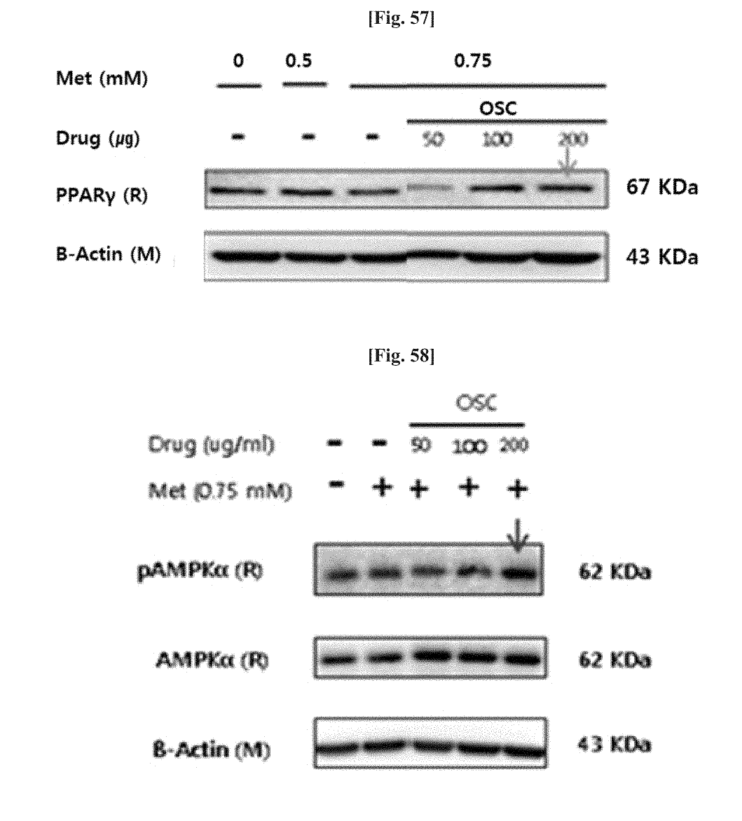

As a result, as illustrated in FIGS. 55 to 58, when compared with groups administered with metformin alone (MET1 and MET2), the expression level of dipeptidyl peptidase-4 (DPP-4) was decreased in a group co-administered with metformin and a Houttuynia cordata extract, whereas the expression levels of PPAR-γ and AMPK were significantly increased in the same. In Example 31, inhibition of the expression of DPP-4, an enzyme responsible for degrading incretin, leads to stimulation of synthesis/secretion of insulin, inhibition of glucagon secretion and inhibition of glucose synthesis in the liver, and thus blood sugar levels can be controlled by regulating the expression of DPP-4. It has been known that increasing PPAR-γ expression has a positive effect on increasing insulin sensitivity, and that AMPK has a central role in regulating energy metabolism and homeostasis in vivo. Accordingly, the results indicate that co-administration of metformin and a Houttuynia cordata extract can further improve an anti-diabetic effect.

### Example 32. Confirmation of Changes in Expression Levels of Related Genes Depending Upon Administration of Houttuynia cordata Extracts

32-1. Preparation of Cells and Houttuynia cordata Extracts

RAW 264.7 cells, a macrophage cell line, were obtained from the Korean Cell Line Bank (KCLB, Seoul, Korea), and DMEM containing 10% FBS, 2 mM L-glutamine, 100 U/ml penicillin, and 100 μg/ml streptomycin was used as a medium for culturing the RAW 264.7 cells. The cells were cultured in a CO₂ incubator set to 37° C. with 5% CO₂ and 95% O₂. Houttuynia cordata extracts (100% water and 30% ethanol extracts) used in the experiments of the present invention were provided from the College of Pharmacy, Dongguk University. Experiments were performed for a total of 4 groups, including a normal group (N), a metformin-administered group (M), a group administered with metformin and a Houttuynia cordata extract (30% ethanol extract) (M+USE), a group administered with metformin and a Houttuynia cordata extract (water extract) (M+USW).

DMEM containing 10% FBS, 2 mM L-glutamine, 100 U/ml penicillin, and 100 μg/ml streptomycin was used as a medium for culturing the RAW 264.7 cells, and the cells were cultured under conditions of 37° C., 5% CO₂, and 90% humidity. The cultured cells were maintained while exchanging the culture medium once every 2 to 3 days. When the cells were fully differentiated, the cell culture was washed with phosphate buffered saline (PBS) and then the cells were detached from a culture dish using a trypsin-EDTA solution. The separated cells were subjected to centrifugation to collect the same, and then the collected cells were mixed with fresh media and used in subculture.

32-2. Expression of Genes Associated with Anti-Diabetic Effect

To confirm whether combined use of metformin and a Houttuynia cordata extract affects the expression of genes associated with an anti-diabetic effect, the gene expression levels of AMPK-α, PPAR-α, and PPAR-γ of RAW 264.7 cells administered with metformin alone (M) and RAW 264.7 cells administered with metformin and a Houttuynia cordata extract (30% ethanol or 100% water) were compared using real-time PCR.

Total RNA was separated and purified using TRIsure (Bioline, USA) according to a protocol. 1 μg of total RNAs was subjected to a reverse transcription reaction using a cDNA synthesis kit (Sprint™RT Complete Oligo-(dT)18, Clontech, Mountain View, Calif., USA) according to a protocol for synthesizing first strand cDNA. The produced RT-PCR sample was subjected to real-time PCR, in which the final reaction volume was adjusted to 20 μl and Light Cycler-Fast Start DNA Master SYBR Green (Roche Applied Science, Indianapolis, Ind., USA) and a Light Cycler instrument (Roche Applied Science) were used.

DNA sequences of primers used in Example 32-2 are as follows.

TABLE 10

| Genus | | | |
|---|---|---|---|
| Specific | | | Annealing |
| Primers | Direction | Sequence | temp. |
| Beta-actin | F | 5′-GCAAGTGCTTCTAGGCGGAC-3′ | 52° C. |
| | | (SEQ ID NO. 1) | |
| | R | 5′-AAGAAAGGGTGTAAAACGCAGC-3′ | |
| | | (SEQ ID NO. 2) | |
| AMPK alpha 1 | F | 5′-AAGCCGACCCAATGACATCA-3′ | 49° C. |
| | | (SEQ ID NO. 3) | |
| | R | 5′-CTTCCTTCGTACACGCAAAT-3′ | |
| | | (SEQ ID NO. 4) | |
| PPAR-alpha | F | 5′-GCCTGTCTGTCGGGATGT-3′ | 50° C. |
| | | (SEQ ID NO. 5) | |
| | R | 5′-GGCTTCGTGGATTCTCTTG-3′ | |
| | | (SEQ ID NO. 6) | |
| PPAR-gamma | F | 5′-GCCCTTTGGTGACTTTATGGA-3′ | 51° C. |
| | | (SEQ ID NO. 7) | |
| | R | 5′-GCAGCAGGTTGTCTTGGATG-3′ | |
| | | (SEQ ID NO. 8) | |

PCR amplification was performed according to PCR steps, consisting of a pre-incubation step at 95° C. for 10 minutes and 35 (for beta-actin) or 45 (for C/EBPa) cycles of amplification (denaturation at 95° C. for 10 seconds, annealing at 52° C. for 10 seconds, and extension at 72° C. for 15 seconds). Total RNA was separated and purified using TRIsure (Bioline, USA) according to a protocol. 1 μg of total RNAs was subjected to a reverse transcription reaction using a cDNA synthesis kit (Sprint™RT Complete Oligo-(dT)18, Clontech, Mountain View, Calif., USA) according to a protocol for synthesizing first strand cDNA. The produced RT-PCR sample was subjected to real-time PCR, in which the final reaction volume was adjusted to 20 μl and Light Cycler-Fast Start DNA Master SYBR Green (Roche Applied Science, Indianapolis, Ind., USA) and a Light Cycler instrument (Roche Applied Science) were used.

As a result, as illustrated in FIG. 59, a normal group (N) and a metformin-administered group (M) showed 0.80 and 0.76 for the gene expression levels of AMPK-α, respectively. A group co-administered with metformin and a Houttuynia cordata extract (30% ethanol) (M+USE) and a group co-administered with metformin and a Houttuynia cordata extract (100% water) (M+USW) exhibited increased gene expression of AMPK-α. In particular, the group (M+USE) exhibited a significant increase in AMPK-α gene expression, showing a value of 0.84.

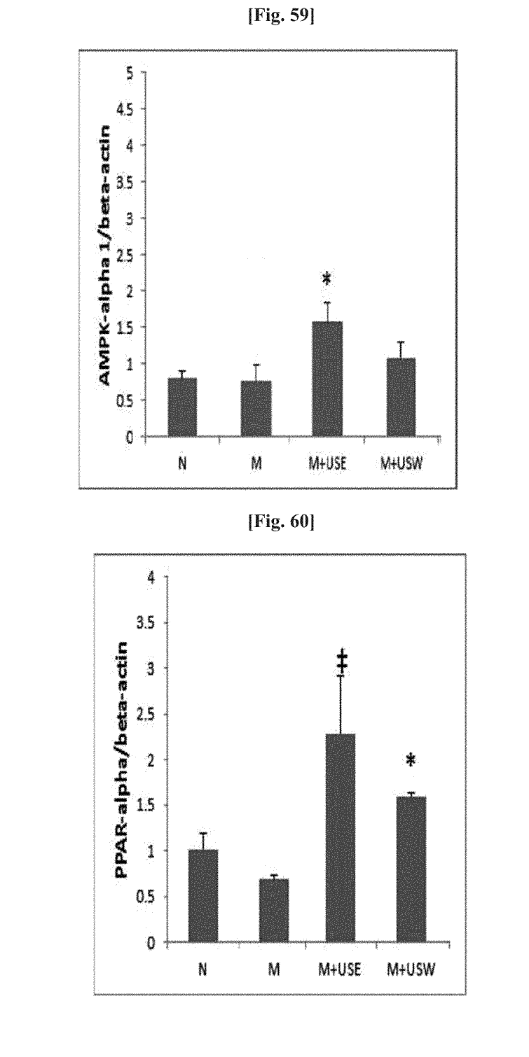

In addition, as illustrated in FIG. 60, a normal group (N) and a metformin-administered group (M) exhibited 1.01 and 0.68 for the gene expression levels of PPAR-α, respectively. A group co-administered with metformin and a Houttuynia cordata extract (30% ethanol) (M+USE) and a group co-administered with metformin and a Houttuynia cordata extract (100% water) (M+USW) exhibited increased gene expression of PPAR-α, showing values of 2.29 and 1.59, respectively.

In addition, as illustrated in FIG. 61, a normal group (N) and a metformin-administered group (M) exhibited 1.03 and 0.83 for the gene expression levels of PPAR-γ, respectively. A group co-administered with metformin and a Houttuynia cordata extract (30% ethanol) (M+USE) exhibited increased gene expression of PPAR-γ, showing a value of 0.94.

32-3. Expression of Genes Associated with Side Effects of Metformin

To identify the effect of co-administration of metformin and a Houttuynia cordata extract on expression of genes, which are associated with side effects caused by metformin, the gene expression levels of XBP-1 of RAW 264.7 cells administered with metformin alone (M) and RAW 264.7 cells administered with metformin and a Houttuynia cordata extract (30% ethanol or 100% water) were compared using real-time PCR. RAW 264.7 cells were harvested according to the same method as described in Example 9-2. Experiments were performed for a total of 4 groups, including a normal group (N), a metformin-administered group (M), a group administered with metformin and a Houttuynia cordata extract (30% ethanol) (M+USE) and a group administered with metformin and a Houttuynia cordata extract (water extract) (M+USW).

To identify the gene expression level of XBP-1, real-time PCR was performed according to the same method as described in Example 10-1 except primers.

DNA sequences of primers used in Example 32-3 are as follows.

TABLE 11

| Genus | | | |
|---|---|---|---|
| Specific | | | Annealing |
| Primers | Direction | Sequence | temp. |
| beta-actin | F | 5′-GCAAGTGCTTCTAGGCGGAC-3′ | 52° C. |
| | | (SEQ ID NO. 1) | |
| | R | 5′-AAGAAAGGGTGTAAAACGCAGC-3′ | |
| | | (SEQ ID NO. 2) | |
| XBP-1 | F | 5′-TGGCCGGGTCTGCTGAGTCCG-3′ | 51° C. |
| | | (SEQ ID NO. 9) | |
| | R | 5′-GTCCATGGGAAGATGTTCTGG-3′ | |
| | | (SEQ ID NO. 10) | |

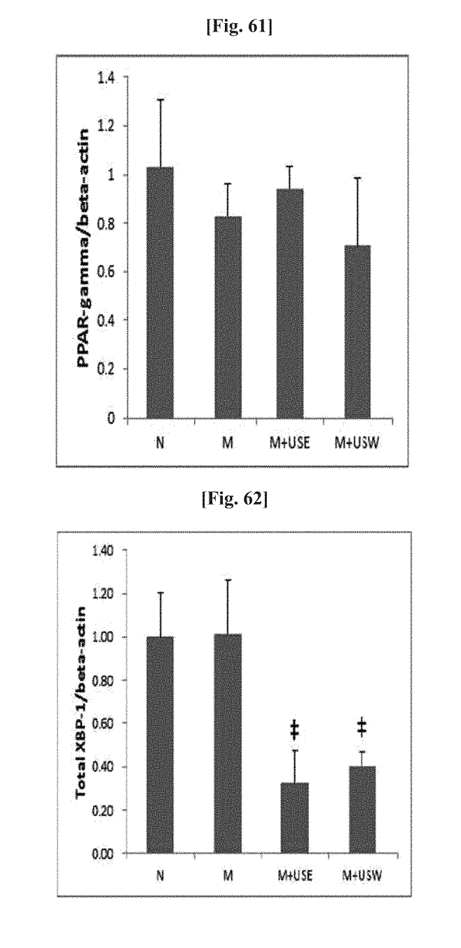

As a result, as illustrated in FIG. 62, a normal group (N) and a metformin-administered group (M) exhibited 1.00 and 1.01 for the gene expression levels of XBP-1, respectively. A group co-administered with metformin and a Houttuynia cordata extract (30% ethanol) (M+USE) and a group co-administered with metformin and a Houttuynia cordata extract (100% water) (M+USW) exhibited decreased gene expression of XBP-1, showing 0.32 and 0.4, respectively.

### Example 33. Intraperitoneal Insulin Tolerance Test (IPITT) According to Administration of Houttuynia cordata Extracts

Using the same method as described in Example 11, an IPITT was performed to identify the effect of administration of Houttuynia cordata extracts on insulin tolerance.

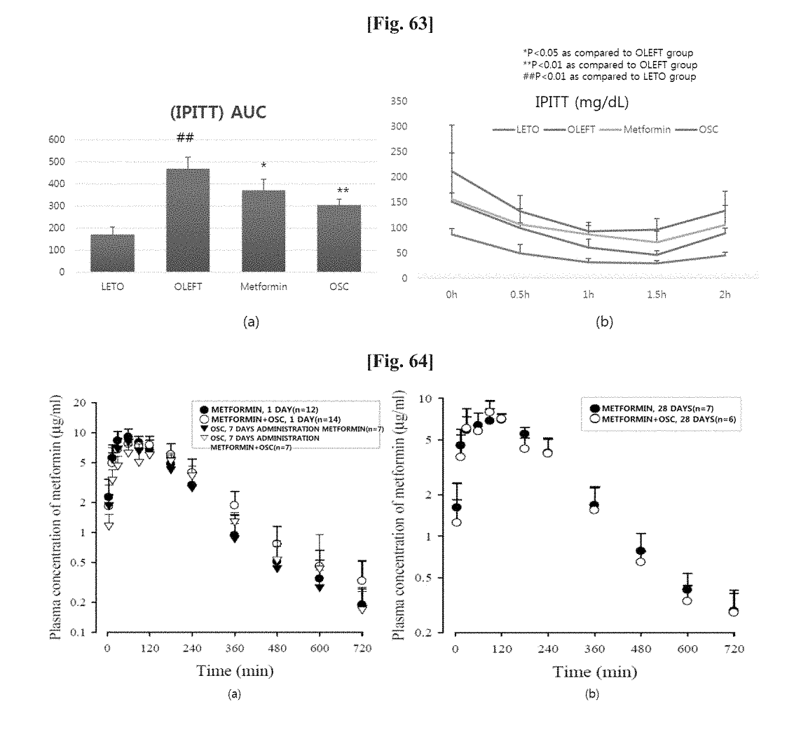

As a result, as illustrated in FIG. 63, an OLEFT group exhibited a higher insulin resistance than a LETO group, and the insulin resistance showed a tendency of decreasing through treatment of metformin alone. In addition, when compared to a group administered with metformin alone, groups co-administered with metformin and a Houttuynia cordata extract exhibited a significant decrease in insulin resistance.

### Example 34. Measurement of Pharmacokinetic Changes of Metformin by Co-Administration of Metformin and Houttuynia cordata Extract

### 34-1. Pharmacokinetic Changes of Metformin According to Period of Co-Administration

Using the same method as described in Example 12-1, pharmacokinetic changes of metformin according to the period of co-administration were measured.

As a result, as illustrated in the following Table 12 and FIG. 64, when compared to a group administered with metformin alone, no significant changes in pharmacokinetic parameters such as the accumulation effect of metformin were observed in a group co-administered with metformin and a Houttuynia cordata extract (once, 7 days, or 4 weeks).

TABLE 12

| | | | Houttuynia cordata, | | Houttuynia cordata, | |
|---|---|---|---|---|---|---|
| | Single administration | | 7 days administration | | 4 weeks administration | |
| | | Metformin + | | Metformin + | | Metformin + |
| | | Houttuynia | | Houttuynia | | Houttuynia |
| | Metformin | cordata | Metformin | cordata | Metformin | cordata |
| Parameter | (n = 12) | (n = 14) | (n = 7) | (n = 7) | (n = 7) | (n = 7) |
| Body | 276 ± 32.8 | 274 ± 31.5 | 284 ± 12.7 | 289 ± 10.7 | 354 ± 34.1 | 338 ± 13.4 |
| weight (g) | | | | | | |
| AUC_{0-720 min} | 1797 ± 297 | 2101 ± 397^{a} | 1616 ± 176 | 1633 ± 179 | 1922 ± 165 | 1808 ± 200 |
| (μg | | | | | | |
| min/ml) | | | | | | |
| AUC_{0-∞} (μg | 1865 ± 309 | 2172 ± 419^{a} | 1657 ± 181 | 1676 ± 202 | 1991 ± 156 | 1880 ± 213 |
| min/ml) | | | | | | |
| Terminal | 174 ± 47.8 | 152 ± 43.5 | 143 ± 41.8 | 118 ± 42.8 | 156 ± 49.5 | 164 ± 58.7 |
| half- | | | | | | |
| life (min) | | | | | | |
| CL/F (ml/min/ | 54.4 ± 9.68 | 48.1 ± 9.62 | 61.0 ± 6.79 | 60.4 ± 7.74 | 50.5 ± 3.90 | 53.9 ± 6.73 |
| kg) | | | | | | |
| Cₘₐₓ (μg/ | 9.49 ± 1.65 | 8.42 ± 1.53 | 8.46 ± 2.49 | 6.71 ± 0.911 | 7.45 ± 0.960 | 8.22 ± 1.11 |
| ml) | | | | | | |
| Tₘₐₓ (min)^{b} | 60 (30-60) | 90 (30-240)^{a} | 60 (30-120) | 90 (60-180) | 120 (60-180) | 90 (90-180) |
| CL_{R} (ml/min/ | 39.1 ± 8.68 | 36.2 ± 7.92 | 50.1 ± 6.21 | 49.1 ± 6.06 | 45.3 ± 6.02 | 43.4 ± 6.81 |
| kg) | | | | | | |
| Ae_{0-24 h} (% | 70.7 ± 14.6 | 75.4 ± 10.2 | 82.7 ± 11.4 | 81.4 ± 5.24 | 89.4 ± 6.21 | 80.5 ± 5.74^{a} |
| of dose) | | | | | | |
| GI_{24 h} (% of | 9.00 ± 4.22 | 6.81 ± 3.47 | 10.3 ± 5.28 | 7.55 ± 4.08 | 11.2 ± 2.39 | 8.43 ± 4.42 |
| dose) | | | | | | |
| AUC_{0-720 min}/ | 96.3 ± 1.22 | 96.8 ± 1.31 | 97.5 ± 1.39 | 97.6 ± 1.90 | 96.5 ± 2.43 | 96.2 ± 2.57 |
| AUC_{0-∞} | | | | | | |
| (%) | | | | | | |

### 34-2. Changes in Metformin Uptake by Inhibition of OCT 1 and OCT 2

Using the same method as described in Example 12-2, changes in metformin uptake by inhibition of OCT 1 and 2 were measured.

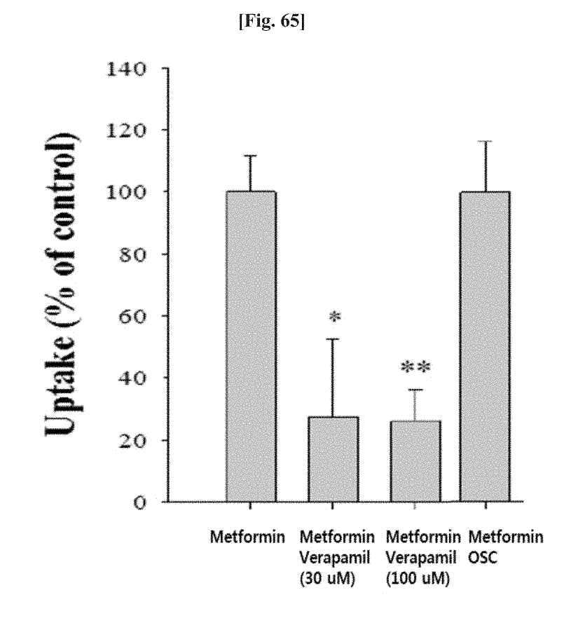

As a result, as illustrated in FIG. 65, when treated with verapamil, an inhibitor of OCT1 and 2, (OCT1: 30 μM verapamil and OCT2: 500 μM verapamil), a significant decrease in metformin uptake was observed in a group administered with metformin alone, whereas no decrease in metformin uptake was observed in a group co-administered with metformin and a Houttuynia cordata extract.

Taken together, it was confirmed that co-administration of a Houttuynia cordata extract and metformin, an anti-diabetic drug, has no effect on absorption and action of metformin drug itself.

The aforementioned description of the present invention is provided by way of example and those skilled in the art will understood that the present invention can be easily changed or modified into other specified forms without change or modification of the technical spirit or essential characteristics of the present invention. Therefore, it should be understood that the aforementioned examples are only provided by way of example and not provided to limit the present invention.

### INDUSTRIAL APPLICABILITY

The present invention relates to a composition for improving anti-diabetic and anti-obesity effects, including an extract extracted from any one selected from the group consisting of Lonicera japonica (Lonicerae Flos), Scutellaria baicalensis (Scutellariae Radix), and Houttuynia cordata (Houttuyniae Herba).

It was confirmed that combined use of the extract of the present invention and metformin, an anti-diabetic drug, improves therapeutic effects on diabetes mellitus and prediabetes and reduces side effects. Thus, it is expected that the extract can be usefully used as a pharmaceutical composition for improving a therapeutic effect on diabetes mellitus. In addition, it was confirmed that the extract exhibits an inhibitory effect on fat accumulation along with the therapeutic effect on diabetes mellitus. Therefore, it is expected that the extract can prevent or treat obesity along with treating diabetes.

1-6. (canceled)

7. A method of improving anti-diabetic effect, the method comprising a step of administering an extract extracted from any one selected from the group consisting of Lonicera japonica (Lonicerae Flos), Scutellaria baicalensis (Scutellariae Radix), and Houttuynia cordata (Houttuyniae Herba) to an individual.

8. A method of treating diabetes mellitus, the method comprising a step of administering an extract extracted from any one selected from the group consisting of Lonicera japonica (Lonicerae Flos), Scutellaria baicalensis (Scutellariae Radix), and Houttuynia cordata (Houttuyniae Herba) to an individual.

9. (canceled)

10. The method according to claim 7, wherein the extract is administrated simultaneously with or separately from the anti-diabetic drug, or the pharmaceutical composition and the anti-diabetic drug are administrated sequentially.

11. The method according to claim 7, wherein the method inhibits differentiation of fat cells.

12. The method according to claim 7, wherein the extract is extracted using one or more solvents selected from the group consisting of water, alcohols having 1 to 4 carbons, and a combination thereof.

13. The method according to claim 7, wherein the method increases expression levels of one or more selected from the group consisting of phosphorylated AMP-activated protein kinase (p-AMPK) and genes encoding sirtuin 1 (SirT1), AMP-activated protein kinase-alpha (AMPK-α), peroxisome proliferator-activated receptor-alpha (PPAR-α), and peroxisome proliferator-activated receptor-gamma (PPAR-γ), respectively.

14. The method according to claim 7, wherein method decreases expression levels of one or more selected from the group consisting of genes encoding X-box binding protein 1 (XBP-1), tumor necrosis factor-alpha (TNF-α), and interleukin-6 (IL-6), respectively.

15. The method according to claim 8, wherein the extract is administrated simultaneously with or separately from the anti-diabetic drug, or the pharmaceutical composition and the anti-diabetic drug are administrated sequentially.

16. The method according to claim 8, wherein the extract inhibits differentiation of fat cells.

17. The method according to claim 8, wherein the extract is extracted using one or more solvents selected from the group consisting of water, alcohols having 1 to 4 carbons, and a combination thereof.

18. The method according to claim 8, wherein the method increases expression levels of one or more selected from the group consisting of phosphorylated AMP-activated protein kinase (p-AMPK) and genes encoding sirtuin 1 (SirT1), AMP-activated protein kinase-alpha (AMPK-α), peroxisome proliferator-activated receptor-alpha (PPAR-α), and peroxisome proliferator-activated receptor-gamma (PPAR-γ), respectively.

19. The method according to claim 8, wherein the method decreases expression levels of one or more selected from the group consisting of genes encoding X-box binding protein 1 (XBP-1), tumor necrosis factor-alpha (TNF-α), and interleukin-6 (IL-6), respectively.


SEQUENCE LISTING (14 sequences)

SEQ ID NO: 1 - DNA, 20 bp, Artificial Sequence
  beta actin primer_forward
gcaagtgcttctaggcggac

SEQ ID NO: 2 - DNA, 22 bp, Artificial Sequence
  beta actin primer_reverse
aagaaagggtgtaaaacgcagc

SEQ ID NO: 3 - DNA, 20 bp, Artificial Sequence
  AMPK alpha primer_forward
aagccgacccaatgacatca

SEQ ID NO: 4 - DNA, 20 bp, Artificial Sequence
  AMPK alpha primer_reverse
cttccttcgtacacgcaaat

SEQ ID NO: 5 - DNA, 18 bp, Artificial Sequence
  PPAR-alpha primer_forward
gcctgtctgtcgggatgt

SEQ ID NO: 6 - DNA, 19 bp, Artificial Sequence
  PPAR-alpha primer_reverse
ggcttcgtggattctcttg

SEQ ID NO: 7 - DNA, 21 bp, Artificial Sequence
  PPAR-gamma primer_forward
gccctttggtgactttatgga

SEQ ID NO: 8 - DNA, 20 bp, Artificial Sequence
  PPAR-gamma primer_reverse
gcagcaggttgtcttggatg

SEQ ID NO: 9 - DNA, 21 bp, Artificial Sequence
  XBP-1 primer_forward
tggccgggtctgctgagtccg

SEQ ID NO: 10 - DNA, 21 bp, Artificial Sequence
  XBP-1 primer_reverse
gtccatgggaagatgttctgg

SEQ ID NO: 11 - DNA, 20 bp, Artificial Sequence
  TNF-alpha primer_forward
gaactggcagaagaggcact

SEQ ID NO: 12 - DNA, 20 bp, Artificial Sequence
  TNF-alpha primer_reverse
agggtctgggccatagaact

SEQ ID NO: 13 - DNA, 20 bp, Artificial Sequence
  IL-6 primer_forward
agttgccttcttgggactga

SEQ ID NO: 14 - DNA, 20 bp, Artificial Sequence
  IL-6 primer_reverse
cagaattgccattgcacaac